(19) United States
(12) Patent Application Publication
(10) Pub. No.: US 2012/0168663 A1
(43) Pub. Date: Jul. 5, 2012

(54) COMPOSITIONS AND METHODS COMPRISING TRIFLUORONITROMETHANE

(75) Inventors: Rajiv R. Singh, Morristown, NJ (US); Michael Van Der Puy, Morristown, NJ (US); Andrew J. Poss, Morristown, NJ (US); Ian R. Shankland, Morristown, NJ (US)

(73) Assignee: HONEYWELL INTERNATIONAL INC., Morristown, NJ (US)

(21) Appl. No.: 13/377,915

(22) PCT Filed: Jun. 15, 2010
(86) PCT No.: PCT/US2010/038695

§ 371 Date: Mar. 22, 2012

Related U.S. Application Data
(60) Provisional application No. 61/187,083, filed on Jun. 15, 2009.

(51) Int. Cl. C07C 205/08; C11D 17/00; C09K 21/10; A62D 1/00; C09K 5/02; C09K 3/00

(52) U.S. Cl. 252/2; 568/946; 252/67; 252/364; 252/601; 510/405; 252/68; 516/12

(57) ABSTRACT

Disclosed are heat transfer fluids which possess a highly desirable and unexpectedly superior combination of properties, and heat transfer systems and methods based on these fluids. The heat transfer fluid comprise from about 30 to about 70 percent, on a molar basis, of carbon dioxide (CO2) and from about 30 to about 70 percent, on a molar basis, of hydrofluorocarbon (HFC), preferably HFC having one to two carbon atoms, and even more preferably trans-1,1,1,3-tetrafluoropropene (HFC-32). The preferred fluids of the present invention have a vapor pressure of at least about 100 psia at 40?F and are also preferably not azeotropic.

### FIELD OF THE INVENTION

This invention relates to compositions and methods which make advantageous use of trifluoronitromethane (CF₃NO₂), and in particular embodiments to heat transfer fluids and heat transfer methods which utilize trifluoronitromethane (CF₃NO₂).

### BACKGROUND

It is desirable in many different situations to selectively transfer heat between a fluid and a body to be cooled or warmed. As used herein, the term “body” refers not only to solid bodies but also other fluid materials which take the shape of the container in which they exist.

One well known system for achieving such transfer of heat achieves cooling of a body by first pressurizing a vapor phase heat transfer fluid and then expanding it through a Joule-Thomson expansion element, such as a valve, orifice, or other type of flow constriction. Any such device will be referred to hereinafter simply as a Joule-Thompson “expansion element,” and systems which use such an element are sometimes referred to herein as Joule-Thompson systems. In most Joule-Thomson systems, single component, non-ideal gasses are pressurized and then expanded through a throttling component or expansion element, to produce substantially isenthalpic cooling. The characteristics of the gas used, such as boiling point, inversion temperature, critical temperature, and critical pressure effect the starting pressure needed to reach a desired cooling temperature. While such characteristics are all generally well known and/or relatively easy to predict with an acceptable degree of certainty for many single component fluids, this is not necessarily the case for multi-component fluids

Because of the large number of properties or characteristics which are relevant to the effectiveness and desirability of a heat transfer fluid and to the heat transfer methods which use such fluids, it is frequently difficult to predict in advance how any particular multi-component fluid will perform as a heat transfer fluid. For example, U.S. Pat. No. 5,774,052—Bivens discloses a combination of difluoroethane (HFC-32), pentafluoroethane (HFC-125) and a small amount (ie., up to 5% by weight) of carbon dioxide (CO2) in the form of an azeotropic fluid that is said to have advantages as a refrigerant in certain applications. The fluids of Bivens are comprised of compounds which are potentially environmentally damaging from a global warming perspective, and using fluids with azeotropic properties can sometimes result in a costly refrigerant.

U.S. Pat. No. 5,763,063—Richard et al. discloses a non-azeotropic combination of various hydrocarbons, including HFC-32, and carbon dioxide which form a fluid said to be acceptable as a replacement for chlorotrans-1,1,1,3-tetrafluoropropene (HCFC-22). In particular, the Richard et al. patent teaches that the vapor pressure of this fluid is substantially equal to HCFC-22, which is only about 83 psia. Therefore, while the fluid of Richard et al. is expected to perform well in certain refrigeration applications, it may be considered inadequate in several other types of heat transfer applications, including the same types of applications mentioned above with respect to the Bivens fluid.

The compound trifluoronitromethane (CF₃NO₂) has been suggested for use in various applications, including the generation of information recording media, gaseous ultrasound contrast media, therapeutic delivery systems, gas and gaseous precursor-filled microspheres. See “New Preparative Routes, Scale-Up, and Properties of Trifluoronitromethane, F3CNO2 and Related Reactions,” Research Seminar, University of Alabama in Apr. 17, 2007. This paper also suggests that this material might be a suitable replacement for the various agents used in refrigeration and fire extinguishing agents, such as the various Halons.

### SUMMARY

Applicants have developed compositions comprising trifluoronitromethane (CF₃NO₂). In certain preferred embodiments, the present compositions are useful as, or in connection with, heat transfer fluids, blowing agents, foams, foamable compositions, foam pre-mixes, solvents, cleaning fluids, extractants, flame retardants, fire suppression agents, deposition agents, propellants, sprayable compositions, deposition agents, and to methods and systems relating to each of these.

The preferred compositions possess a highly desirable yet difficult to obtain combination of properties. The combination of properties possessed by the present compositions is important in many applications. For example, particularly in heat transfer applications but for other applications as well, the following combination of properties and characteristics is highly desirable and possessed by the preferred compositions: chemical stability, low toxicity, low- or non-flammability, and efficiency in-use, while at the same time substantially reducing or eliminating the deleterious ozone depletion potential of many of the compositions, such as refrigerants, which have heretofore been commonly used, such as CFCs. In addition, the preferred embodiments of the present invention provide compositions, particularly and preferably in certain embodiments heat transfer fluids such as refrigerants, which also substantially reduce or eliminate the negative global warming effects associated with previously used heat transfer fluids. Certain of the preferred heat transfer compositions of the present invention which comprise trifluoronitromethane and at least one co-refrigerant provide a relatively high refrigeration capacity and/or coefficient of performance, in addition to the other desirable properties mentioned above. This difficult to achieve combination of properties and/or characteristics is important in many applications, including particularly by way of example, in low temperature air conditioning, refrigeration and heat pump applications.

In one aspect, the present invention provides a composition comprising trifluoronitromethane (CF₃NO₂) and at least one co-agent. In certain preferred embodiments the present compositions comprise from about 1 to about 99 percent of trifluoronitromethane (CF₃NO₂) and from about 1 to about 99 percent of at least one co-agent. Unless otherwise specified herein, reference to percentages refers to weight percent. In certain preferred embodiments, the compositions comprise from about 40 to about 99 percent of CF₃NO₂ and from about 1 to about 60 percent of at least one co-agent. In certain highly preferred embodiments, the at least one co-agent is selected from the following group: carbon dioxide (CO₂); tetrafluoropropenes, including 2,3,3,3-tetrafluoropropene (HFO-1234yf) and 1,3,3,3-tetrafluoropropene (HFO-1234ze); C1-C4 hydrocarbons, including preferably C3 and C4 hydrocarbons; hydrofluorocarbons (HFCs), including preferably difluoromethane (HFC-32); difluoroethane (HFC-152a); 1,1,1,2-tetrafluoroethane (HFC-134a); and pentafluoroethane (HFC-125); ammonia; and combinations of any two or more of these.

As used herein, the term “co-agent” is used for the purposes of convenience but not by way of limitation to refer to any compound, other than CF₃NO₂, which is present in the composition and which participates in the function of the composition for its intended purpose. In certain preferred embodiments, therefore, the co-agent is a compound, or combination of compounds, which act in the composition as a co-refrigerant, co-blowing agent, co-solvent, co-cleaner, co-deposition agent, co-extractant, co-fire suppressant, co-fire extinguishing agent or co-propellant.

In one aspect, the present invention provides compositions, and preferably heat transfer fluids, comprising CF₃NO₂ and at least one co-refrigerant. In certain preferred embodiments the present compositions, particularly heat transfer fluids, comprise from about 40 to about 99 percent of CF₃NO₂ and from about 1 to about 60 percent of at least one co-refrigerant. In certain highly preferred embodiments, the at least one co-refrigerant is selected from the group carbon dioxide (CO₂), 2,3,3,3-tetrafluoropropene (HFO-1234yf), 1,3,3,3-tetrafluoropropene (HFO-1234ze), C1-C4 hydrocarbons, and combinations of any two or more of these.

As with the co-agents of the present compositions in general, it is contemplated that the co-refrigerant may include compounds other than and/or in addition to carbon dioxide (CO₂), 2,3,3,3-tetrafluoropropene (HFO-1234yf), 1,3,3,3-tetrafluoropropene (HFO-1234ze), C1-C4 hydrocarbons, and combinations of any two or more of these. In certain preferred embodiments, the co-refrigerant is selected from the group consisting of carbon dioxide (CO₂), 2,3,3,3-tetrafluoropropene (HFO-1234yf), 1,3,3,3-tetrafluoropropene (HFO-1234ze), C1-C4 hydrocarbons, and combinations of any two or more of these.

As used herein, the term “co-refrigerant” is used for the purposes of convenience but not by way of limitation to refer to any compound, other than CF₃NO₂, which is present in the composition for the purpose of contributing to and/ or otherwise participating in the heat transfer characteristics of the composition or for the purpose of being involved in the transfer of heat, and is specifically intended to include such compound(s) which are present when the heat transfer involves heating and/or cooling or refrigeration.

As used herein, the term C1-C4 hydrocarbons is used in its broad sense to include all hydrocarbons, whether branched or unbranched, having at least one and not more than four carbon atoms in a molecule.

In certain preferred embodiments, the heat transfer fluids preferably comprise from about 60 to about 99 percent CF₃NO₂ and from about 1 to about 40 percent of at least one co-refrigerant comprising, and in certain embodiments consisting essentially of, carbon dioxide (CO₂). In other embodiments, the heat transfer fluids preferably comprise from about 70 to about 95 percent by weight of CF₃NO₂ and from about 5 to about 30 percent of at least one co-refrigerant, preferably comprising, and in certain embodiments consisting essentially of, carbon dioxide (CO₂). The preferred fluids of the present invention which comprise CO₂ have a vapor pressure of at least about 30 psia at 35° F.

In certain preferred embodiments, the heat transfer fluids preferably comprise from about 40 to about 99 percent CF₃NO₂ and from about 1 to about 60 percent by weight of at least one co-refrigerant comprising, and in certain embodiments consisting essentially of 2,3,3,3-tetrafluoropropene (HFO-1234yf). In other embodiments, the heat transfer fluids preferably comprise from about 60 to about 95 percent CF₃NO₂ and from about 5 to about 40 percent by weight of at least one co-refrigerant, preferably comprising, and in certain embodiments consisting essentially of, 2,3,3,3-tetrafluoropropene (HFO-1234yf).

In certain preferred embodiments, the heat transfer fluids preferably comprise from about 40 to about 99 percent CF₃NO₂ and from about 1 to about 60 percent of at least one co-refrigerant comprising, and in certain embodiments consisting essentially of 1,3,3,3-tetrafluoropropene (HFO-1234ze). In other embodiments, the heat transfer fluids preferably comprise from about 60 to about 95 percent CF₃NO₂ and from about 5 to about 40 percent of at least one co-refrigerant, preferably comprising, and in certain embodiments consisting essentially of, 1,3,3,3-tetrafluoropropene (HFO-1234ze). As used herein, the terms 1,3,3,3-tetrafluoropropene HFO-1234ze ar used broadly to encompass all stereoisomeric versions thereof, including cis- and trans- versions of this compound in all relative percentages ranging from 100% cis to 100% trans and all percentages in between.

In certain preferred embodiments, the heat transfer fluids preferably comprise from about 40 to about 99 percent CF₃NO₂ and from about 1 to about 60 percent of at least one co-refrigerant comprising, and in certain embodiments consisting essentially of at least one C1-C4 hydrocarbon, preferably C3-C4 hydrocarbons such as propane, isobutane, n-butane and the like. In other embodiments, the heat transfer fluids preferably comprise from about 60 to about 95 percent CF₃NO₂ and from about 5 to about 40 percent of at least one co-refrigerant, preferably comprising, and in certain embodiments consisting essentially of, at least one C1-C4 hydrocarbon.

The preferred fluids of the present invention are not azeotropic.

According to certain preferred embodiments, the present compositions may further comprise a lubricant, preferably in an amount of from about 1 to 50% by weight of the composition. It is contemplated that those skilled in the art will be able to select, in view of the teachings contained herein, the appropriate lubricant, or combination of lubricants, to use in any given application, and all such lubricants are within the broad scope of the present invention. In certain preferred embodiments, the present compositions, particularly the present heat transfer fluids, comprise one or more lubricants selected from polyol esters (POEs), capped or uncapped polyalkylene glycols (PAGs), mineral oils, silicone oils, polyvinyl ethers (PVE) oils, and the like, and combinations of any two or more of these. All lubricants which are presently well known lubricants or which hereafter become well known lubricants in the refrigeration industry are believed to be adaptable for use in accordance with the present compositions and methods. In certain preferred embodiments, the present compositions comprise one or more lubricants soluble in trifluoronitromethane (CF₃NO₂), and even more preferably soluble in the combination of CF₃NO₂ and co-refrigerant, in amounts of up to about 10% at at least one temperature between from about −40 to about +60 C.

### DETAILED DESCRIPTION OF PREFERRED EMBODIMENTS

In certain preferred forms, the present compositions have a Global Warming Potential (GWP) of not greater than about 1500, more preferably not greater than about 1000, more preferably not greater than about 500, and even more preferably not greater than about 150. In certain embodiments, the GWP is not greater than about 100 and even more preferably not greater than about 75. As used herein, “GWP” is measured relative to that of carbon dioxide and over a 100 year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

The amount of the CF₃NO₂ contained in the present compositions can vary widely, depending the particular application, and compositions containing more than trace amounts and less than 100% of the compound are within broad the scope of the present invention, although it should be understood that various use and method aspects of the present invention are adaptable for use of CF₃NO₂ at essentially 100 percent of the composition. In preferred embodiments, the present compositions, particularly blowing agent and heat transfer compositions, comprise CF₃NO₂ in amounts from about 5% to about 99%, and even more preferably from about 5% to about 95%.

Many additional compounds or components, including lubricants, stabilizers, metal passivators, corrosion inhibitors, flammability suppressants, and other compounds and/or components that modulate a particular property of the compositions (such as cost for example) may be included in the present compositions, and the presence of all such compounds and components is within the broad scope of the invention. In certain preferred embodiments, the present compositions include, in addition to trifluoronitromethane (CF₃NO₂), one or more of the following:

1. Trichlorofluoromethane (CFC-11);

2. Dichlorodifluoromethane (CFC-12);

3. Difluoromethane (HFC-32);

4. Pentafluoroethane (HFC-125);

5. 1,1,2,2-tetrafluoroethane (HFC-134);

6. 1,1,1,2-Tetrafluoroethane (HFC-134a);

7. Difluoroethane (HFC-152a);

8. 1,1,1,2,3,3,3-Heptafluoropropane (HFC-227ea);

9. 1,1,1,3,3,3-hexafluoropropane (HFC-236fa);

10. 1,1,1,3,3-pentafluoropropane (HFC-245fa);

11. 1,1,1,3,3-pentafluorobutane (HFC-365mfc);

12. water; and

13. CO₂

The relative amount of any of the above noted compounds of the present invention, as well as any additional components which may be included in present compositions, can vary widely within the general broad scope of the present invention according to the particular application for the composition, and all such relative amounts are considered to be within the scope hereof.

Accordingly, applicants have recognized that certain compositions of the present invention can be used to great advantage in a number of applications. For example, included in the present invention are methods and compositions relating to heat transfer applications, foam and blowing agent applications, propellant applications, sprayable composition applications, sterilization applications, aerosol applications, compatibilizer applications, fragrance and flavor applications, solvent applications, cleaning applications, inflating agent applications and others. It is believed that those of skill in the art will be readily able to adapt the present compositions for use in any and all such applications without undue experimentation.

The present compositions are generally useful as replacements for CFCs, such as dichlorodifluormethane (CFC-12), HCFCs, such as chlorodifluoromethane (HCFC-22), HFCs, such as tetrafluoroethane (HFC-134a), and combinations of HFCs and CFCs, such as the combination of CFC-12 and 1,1-difluorethane (HFC-152a) (the combination CFC-12:HFC-152a in a 73.8:26.2 mass ratio being known as R-500) in refrigerant, aerosol, and other applications.

The Heat Transfer Fluids

While in certain embodiments the heat transfer fluids of the present invention consist essentially of CF₃NO₂, in many preferred embodiments the present heat transfer fluids comprise CF₃NO₂ and one or more co-heat transfer agents, preferably in certain embodiments comprising one or more of halogenated olefins, including HFO-1234yf, HFO-1234ze and combinations thereof, hydrocarbons, hydrofluorocarbons, including HFC-134a and HFC-32, and combinations of therse, CO₂, and combinations of any two or more of these.

The heat transfer fluids of the present invention are adaptable for use in a wide variety of heat transfer applications, and all such applications are within the scope of the present invention. The present fluids find particular advantage and unexpectedly beneficial properties in connection with applications that require and/or can benefit from the use of highly efficient, non-flammable refrigerants that exhibit low or negligible global warming effects, and low or no ozone depletion potential. The present fluids also provide advantage to low temperature refrigeration applications, such as those in which the refrigerant is provided at a temperature of about −20° C. or less and which have relatively high cooling power.

In certain embodiments, the preferred heat transfer fluids are highly efficient in that they exhibit a coefficient of performance (COP) that is high relative to the COP of the individual components of the fluid and/or relative to many refrigerants which have previously been used. The term COP is well known to those skilled in the art and is based on the theoretical performance of a refrigerant at specific operating conditions as estimated from the thermodynamic properties of the refrigerant using standard refrigeration cycle analysis techniques. See, for example, “Fluorocarbons Refrigerants Handbook”, Ch. 3, Prentice-Hall, (1988), by R. C. Downing, which is incorporated herein by reference. The coefficient of performance, COP, is a universally accepted measure, especially useful in representing the relative thermodynamic efficiency of a refrigerant in a specific heating or cooling cycle involving evaporation or condensation of refrigerant. COP is related to or a measure of the ratio of useful refrigeration to the energy applied by the compressor in compressing the vapor and therefore expresses the capability of a given compressor to pump quantities of heat for a given volumetric flow rate of a heat transfer fluid, such as a refrigerant. In other words, given a specific compressor, a refrigerant with a higher COP will deliver more cooling or heating power. In certain embodiments, the preferred heat transfer fluids exhibit a capacity that is high relative to the capacity of the individual components of the fluid and/or relative to many refrigerants which have previously been used. The cooling capacity of a refrigerant is also an important parameter and can be estimated from certain of the thermodynamic properties of the refrigerant. If the refrigerant is to be used in a system designed for another refrigerant, it is preferred that the capacity of the two refrigerants are similar in order to obtain a similar performance with the same equipment and equipment design. Among the common refrigerants being used in refrigeration and air conditioning/ heat pumps, and which may be replaced by the preferred refrigerants of the present invention with a desirable and advantageous match to COP and/or capacity are: R-134a, R-507A, R-404A, R-22, R-407C and R-410A. The applicants have found that various composition of this invention can be used in the applications where these refrigerants are used with slight adjustments in composition.

As mentioned before, additional components known to those skilled in the art may be added to the mixture to tailor the properties of the heat transfer fluid according to the need.

In connection with evaporative cooling applications, the present compositions are brought in contact, either directly or indirectly, with a body to be cooled and thereafter permitted to evaporate or boil while in such contact, with the preferred result that the boiling gas absorbs heat from the body to be cooled. In such applications it may be preferred to utilize the present compositions, preferably in liquid form, by spraying or otherwise applying the liquid to the body to be cooled. In other evaporative cooling applications, it may be preferred to permit the liquid composition to escape from a relatively high pressure container into a relatively lower pressure environment wherein the body to be cooled is in contact, either directly or indirectly, with the container enclosing the liquid composition of the present invention, preferably without recovering or recompressing the escaped gas. One particular application for this type of embodiment is the self cooling of a beverage, food item, novelty item or the like. Previous to the invention described herein, prior compositions, such as HFC-152a and HFC-134a, were used for such applications. However, such compositions have recently been looked upon negatively in such application because of the negative environmental impact caused by release of these materials into the atmosphere. For example, the United States EPA has determined that the use of such prior chemicals in this application is unacceptable due to the high global warming nature of these chemicals and the resulting detrimental effect on the environment that may result from their use. The compositions of the present invention should have a distinct advantage in this regard due to their low global warming potential and low ozone depletion potential, as described herein. Additionally, the present compositions are expected to also find substantial utility in connection with the cooling of electrical or electronic components, either during manufacture or during accelerated lifetime testing. In a accelerated lifetime testing, the component is sequentially heated and cooled in rapid succession to simulate the use of the component. Such uses would therefore be of particular advantage in the semiconductor and computer board manufacturing industry. Another advantage of the present compositions in this regard is they are expected to exhibit desirable electrical properties when used in connection with such applications. Another evaporative cooling application comprises methods for temporarily causing a discontinuation of the flow of fluid through a conduit. Preferably, such methods would include contacting the conduit, such as a water pipe through which water is flowing, with a liquid composition according to the present invention and allowing the liquid composition of the present invention to evaporate while in contact with the conduit so as to freeze liquid contained therein and thereby temporarily stop the flow of fluid through the conduit. Such methods have distinct advantage in connection with enabling the service or other work to be performed on such conduits, or systems connected to such conduits, at a location downstream of the location at which the present composition is applied.

It is contemplated that the present compositions may include many compounds in widely ranging amounts. It is generally preferred that the present refrigerant compositions comprise CF₃NO₂ in an amount that is at least about 50%, and even more preferably at least about 70% of the composition.

In certain embodiments, it is preferred that the heat transfer compositions comprise at least about 90% CF₃NO₂, more preferably at least about 95% CF₃NO₂, and even more preferably at least about 99% CF₃NO₂.

The relative amount of the hydrofluoroolefin used in accordance with the present invention is preferably selected to produce a heat transfer fluid which has the required heat transfer capacity, particularly refrigeration capacity, and preferably is at the same time non-flammable. As used herein, the term non-flammable refers to a fluid which is non-flammable in all proportions in air as measured by ASTM E-681.

The present compositions may include other components for the purpose of enhancing or providing certain functionality to the composition, or in some cases to reduce the cost of the composition. For example, preferred refrigerant compositions, especially those used in vapor compression systems, include a lubricant, generally in amounts of from about 30 to about 50 percent of the composition. The compositions may also include a co-refrigerant, or compatibilzer, such as propane, for the purpose of aiding compatibility and/or solubility of the lubricant. Such compatibilizers, including propane, butanes and pentanes, are preferably present in amounts of from about 0.5 to about 5 percent of the composition. Combinations of surfactants and solubilizing agents may also be added to the present compositions to aid oil solubility, as disclosed by U.S. Pat. No. 6,516,837, the disclosure of which is incorporated by reference. Commonly used refrigeration lubricants such as Polyol Esters (POEs) and Poly Alkylene Glycols (PAGs), PAG oils, silicone oil, mineral oil, alkyl benzenes (ABs) and poly(alpha-olefin) (PAO) that are used in refrigeration machinery with hydrofluorocarbon (HFC) refrigerants may be used with the refrigerant compositions of the present invention. Commercially available mineral oils include Witco LP 250 (registered trademark) from Witco, Zerol 300 (registered trademark) from Shrieve Chemical, Sunisco 3GS from Witco, and Calumet R015 from Calumet. Commercially available alkyl benzene lubricants include Zerol 150 (registered trademark). Commercially available esters include neopentyl glycol dipelargonate, which is available as Emery 2917 (registered trademark) and Hatcol 2370 (registered trademark). Other useful esters include phosphate esters, dibasic acid esters, and fluoroesters. In some cases, hydrocarbon based oils are have sufficient solubility with the refrigerant that is comprised of an iodocarbon, the combination of the iodocarbon and the hydrocarbon oil might more stable than other types of lubricant. Such combination may therefore be advantageous. Preferred lubricants include polyalkylene glycols and esters. Polyalkylene glycols are highly preferred in certain embodiments because they are currently in use in particular applications such as mobile air-conditioning. Of course, different mixtures of different types of lubricants may be used.

In certain preferred embodiments, the heat transfer composition comprises from about 10% to about 95% CF₃NO₂, and from about 5% to about 90% by weight of an adjuvant, particular in certain embodiments a co-refrigerant (such as HFC-152, HFC-125 and/or CF₃I). The use of the term co-refrigerant is not intended for use herein in a limiting sense regarding the relative performance of CF₃NO₂, but is used instead to identify other components that contribute to the desirable heat transfer characteristics of the composition for a desired application. In certain of such embodiments the co-refrigerant comprises, and preferably consists essentially of, one or more HFCs and/or one or more fluoroiodo C1-C3 compounds, such as trifluroiodomethane, and combinations of these with each other and with other components.

In preferred embodiments in which the co-refrigerant comprises HFC, preferably HFC-125, the composition comprises HFC in an amount of from about 50% to about 95% of the total heat transfer composition, more preferably from about 60% to about 90%, and even more preferably of from about 70% to about 90% of the composition. In such embodiments the present composition preferably comprises, and even more preferably consists essentially of, CF₃NO₂ in an amount of from about 5% to about 50% of the total heat transfer composition, more preferably from about 10% to about 40%, and even more preferably of from about 10% to about 30% of the composition.

The Methods and Systems

The method aspects of the present invention comprise transferring heat to or from a body using a heat transfer fluid in accordance with the present invention. Those skilled in the art will appreciate that many known methods may adapted for use with the present invention in view of the teachings contained herein, and all such methods are within the broad scope hereof. For example, vapor compressions cycles are methods commonly used for refrigeration and/or air conditioning. In its simplest form, the vapor compression cycle involves providing the present heat transfer fluid in liquid form and changing the refrigerant from the liquid to the vapor phase through heat absorption, generally at relatively low pressure, and then from the vapor to the liquid phase through heat removal, generally at an elevated pressure. In such embodiments, the refrigerant of the present invention is vaporized in one or more vessels, such as an evaporator, which is in contact, directly or indirectly, with the body to be cooled. The pressure in the evaporator is such that vaporization of the heat transfer fluid takes place at a temperature below the temperature of the body to be cooled. Thus, heat flows from the body to the refrigerant and causes the refrigerant to vaporize. The heat transfer fluid in vapor form is then removed, preferably by means of a compressor or the like which at once maintains a relatively low pressure in the evaporator and compresses the vapor to a relatively high pressure. The temperature of the vapor is also generally increased as a result of the addition of mechanical energy by the compressor. The high pressure vapor then passes to one or more vessels, preferably a condenser, whereupon heat exchange with a lower temperature medium removes the sensible and latent heats, producing subsequent condensation. The liquid refrigerant, optionally with further cooling, then passes to the expansion valve and is ready to cycle again.

In one embodiment, the present invention provides a method for transferring heat from a body to be cooled to the present heat transfer fluid comprising compressing the fluid in a centrifugal chiller, which may be single or multi-stage. As used herein, the term “centrifugal chiller” refers to one or more pieces of equipment which cause an increase in the pressure of the present heat transfer fluid.

The present methods also provide transferring energy from the heat transfer fluid to a body to be heated, for example, as occurs in a heat pump, which may be used to add energy to the body at a higher temperature. Heat pumps are considered reverse cycle systems because for heating, the operation of the condenser is generally interchanged with that of the refrigeration evaporator.

The present invention also provides methods, systems and apparatus for cooling of objects or very small portions of objects to very low temperatures, sometimes referred to herein for the purposes of convenience, but not by way of limitation, as micro-freezing. The objects to be cooled in accordance with the present micro-freezing methods may include biological matter, electronic components, and the like. In certain embodiments, the invention provides for selective cooling of a very small or even microscopic object to a very low temperature without substantially affecting the temperature of surrounding objects. Such methods, which are sometimes referred to herein as “selective micro-freezing,” are advantageous in several fields, such as for example in electronics, where it may be desirable to apply cooling to a miniature component on a circuit board without substantially cooling adjacent components. Such methods may also provide advantage in the field of medicine, where it may be desirable cool miniature discrete portions of biological tissue to very low temperatures in the performance of cryosurgery, without substantially cooling adjacent tissues.

The present methods, systems and compositions are thus adaptable for use in connection with a wide variety of heat transfer systems in general and refrigeration systems in particular, such as air-conditioning (including both stationary and mobile air conditioning systems), refrigeration, heat-pump systems, and the like. In certain preferred embodiments, the compositions of the present invention are used in refrigeration systems originally designed for use with an HFC refrigerant, such as, for example, HFC-134a, or an HCFC refrigerant, such as, for example, HCFC-22. The preferred compositions tend to exhibit many of the desirable characteristics of HFC-134a and other HFC refrigerants, including a GWP that is as low, or lower than that of conventional HFC refrigerants and a capacity that is as high or higher than such refrigerants and a capacity that is substantially similar to or substantially matches, and preferably is as high as or higher than such refrigerants. Applicants have recognized that certain preferred compositions tend to exhibit relatively low global warming potentials (“GWPs”), preferably less than about 1000, more preferably less than about 500, and even more preferably less than about 150. The relatively constant boiling nature of certain of the present compositions makes them even more desirable than certain conventional HFCs, such as R-404A or combinations of HFC-32, HFC-125 and HFC-134a (the combination HFC-32:HFC-125:HFC134a in approximate 23:25:52 weight ratio is referred to as R-407C), for use as refrigerants in many applications, particularly as replacements for HFC-134, HFC-152a, HFC-22, R-12 and R-500.

In certain other preferred embodiments, the present compositions are used in refrigeration systems originally designed for use with a CFC-refrigerant. Preferred refrigeration compositions of the present invention may be used in refrigeration systems containing a lubricant used conventionally with CFC-refrigerants, such as mineral oils, polyalkylbenzene, polyalkylene glycol oils, and the like, or may be used with other lubricants traditionally used with HFC refrigerants. As used herein the term “refrigeration system” refers generally to any system or apparatus, or any part or portion of such a system or apparatus, which employs a refrigerant to provide cooling. Such refrigeration systems include, for example, air conditioners, electric refrigerators, chillers (including chillers using centrifugal compressors), transport refrigeration systems, commercial refrigeration systems and the like.

Many existing refrigeration systems are currently adapted for use in connection with existing refrigerants, and the compositions of the present invention are believed to be adaptable for use in many of such systems, either with or without system modification. Many applications the compositions of the present invention may provide an advantage as a replacement in smaller systems currently based on certain refrigerants, for example those requiring a small refrigerating capacity and thereby dictating a need for relatively small compressor displacements. Furthermore, in embodiments where it is desired to use a lower capacity refrigerant composition of the present invention, for reasons of efficiency for example, to replace a refrigerant of higher capacity, such embodiments of the present compositions provide a potential advantage. Thus, it is preferred in certain embodiments to use compositions of the present invention, particularly compositions comprising a substantial proportion of, and in some embodiments consisting essentially of the present compositions, as a replacement for existing refrigerants, such as : HFC-134a; CFC-12; HCFC-22; HFC-152a; combinations of pentfluoroethane (HFC-125), trifluorethane (HFC-143a) and tetrafluoroethane (HFC-134a) (the combination HFC-125:HFC-143a:HFC134a in approximate 44:52:4 weight ratio is referred to as R-404A); combinations of HFC-32, HFC-125 and HFC-134a (the combination HFC-32:HFC-125:HFC134a in approximate 23:25:52 weight ratio is referred to as R-407C); combinations of methylene fluoride (HFC-32) and pentfluoroethane (HFC-125) (the combination HFC-32:HFC-125 in approximate 50:50 weight ratio is referred to as R-410A); the combination of CFC-12 and 1,1-difluorethane (HFC-152a) (the combination CFC-12:HFC-152a in a 73.8:26.2 weight ratio is referred to R-500); and combinations of HFC-125 and HFC-143a (the combination HFC-125:HFC143a in approximate 50:50 weight ratio is referred to as R-507A).

In certain embodiments it may also be beneficial to use the present compositions in connection with the replacement of refrigerants formed from the combination HFC-32:HFC-125:HFC134a in approximate 20:40:40 weight ratio, which is referred to as R-407A, or in approximate 15:15:70 weight ratio, which is referred to as R-407D. The present compositions are also believed to be suitable as replacements for the above noted compositions in other applications, such as aerosols, blowing agents and the like, as explained elsewhere herein.

In certain applications, the refrigerants of the present invention potentially permit the beneficial use of larger displacement compressors, thereby resulting in better energy efficiency than other refrigerants, such as HFC-134a. Therefore the refrigerant compositions of the present invention provide the possibility of achieving a competitive advantage on an energy basis for refrigerant replacement applications, including automotive air conditioning systems and devices, commercial refrigeration systems and devices, chillers, residential refrigerator and freezers, general air conditioning systems, heat pumps and the like.

Many existing refrigeration systems are currently adapted for use in connection with existing refrigerants, and the compositions of the present invention are believed to be adaptable for use in many of such systems, either with or without system modification. In many applications the compositions of the present invention may provide an advantage as a replacement in systems which are currently based on refrigerants having a relatively high capacity. Furthermore, in embodiments where it is desired to use a lower capacity refrigerant composition of the present invention, for reasons of cost for example, to replace a refrigerant of higher capacity, such embodiments of the present compositions provide a potential advantage. Thus, it is preferred in certain embodiments to use compositions of the present invention, particularly compositions comprising a substantial proportion of, and in some embodiments consisting essentially of trifluoronitromethane (CF₃NO₂) as a replacement for existing refrigerants, such as HFC-134a. In certain applications, the refrigerants of the present invention potentially permit the beneficial use of larger displacement compressors, thereby resulting in better energy efficiency than other refrigerants, such as HFC-134a. Therefore the refrigerant compositions of the present invention provide the possibility of achieving a competitive advantage on an energy basis for refrigerant replacement applications.

It is contemplated that the compositions of the present also have advantage (either in original systems or when used as a replacement for refrigerants such as CFC-11, CFC-12, HCFC-22, HFC-134a, HFC-152a, R-500 and R-507A), in chillers typically used in connection with commercial air conditioning systems. In certain of such embodiments it is preferred to include in the present compositions from about 0.5 to about 30% of a supplemental flammability suppressant , and in certain cases more preferably 0.5% to about 15% by weight and even more preferably from about 0.5 to about 10% on a weight basis

The present compositions may be used as propellants in sprayable compositions, either alone or in combination with known propellants. The propellant composition comprises, more preferably consists essentially of, and, even more preferably, consists of a composition of the invention. The active ingredient to be sprayed together with inert ingredients, solvents, and other materials may also be present in the sprayable mixture. Preferably, the sprayable composition is an aerosol. Suitable active materials to be sprayed include, without limitation, cosmetic materials such as deodorants, perfumes, hair sprays, cleansers, and polishing agents as well as medicinal materials such as anti-asthma and anti-halitosis medications.

Blowing Agents, Foams and Foamable Compositions

Blowing agents may also comprise or constitute one or more of the present compositions. As mentioned above, the present compositions for use as blowing agents comprise CF₃NO₂, preferably in an amount that is at least about 5%, and even more preferably at least about 15% of the blowing agent. In certain preferred embodiments, the blowing agent comprises at least about 50% of CF₃NO₂,, and in certain embodiments the blowing agent consists essentially of CF₃NO₂. In certain preferred embodiments, the blowing agent of the present invention include, in addition to CF₃NO₂, one or more of co-blowing agents, fillers, vapor pressure modifiers, flame suppressants, stabilizers and like adjuvants. The co-blowing agent can comprise a physical blowing agent, a chemical blowing agent (which preferably in certain embodiments comprises water) or a blowing agent having a combination of physical and chemical blowing agent properties. It will also be appreciated that the blowing agents included in the present compositions, including CF₃NO₂ as well as the co-blowing agent, may exhibit properties in addition to those required to be characterized as a blowing agent. For example, it is contemplated that the blowing agent may include components, including CF₃NO₂, which also impart some beneficial property to the blowing agent composition or to the foamable composition to which it is added. For example, it is within the scope of the present invention for CF₃NO₂ or for the co-blowing agent to also act as a polymer modifier or as a viscosity reduction modifier.

By way of example, one or more of the following components may be included in certain preferred blowing agents of the present invention in widely varying amounts: hydrocarbons, hydrofluorocarbons (HFCs), ethers, alcohols, aldehydes, ketones, methyl formate, formic acid, water, trans-1,2-dichloroethylene, carbon dioxide and combinations of any two or more of these. Among ethers, it is preferred in certain embodiments to use ethers having from one to six carbon atoms. Among alcohols, it is preferred in certain embodiments to use alcohols having from one to four carbon atoms. Among aldehydes, it is preferred in certain embodiments to use aldehydes having from one to four carbon atoms.

In other embodiments, the invention provides foamable compositions. The foamable compositions of the present invention generally include one or more components capable of forming foam having. In certain embodiments, the one or more components comprise a thermosetting composition capable of forming foam and/or foamable compositions. Examples of thermosetting compositions include polyurethane and polyisocyanurate foam compositions, and also phenolic foam compositions. With respect to foam types, particularly polyurethane foam compositions, the present invention provides rigid foam (both closed cell, open cell and any combination thereof), flexible foam, and semiflexible foam, including integral skin foams. The present invention provides also single component foams, which include sprayable single component foams.

The reaction and foaming process may be enhanced through the use of various additives such as catalysts and surfactant materials that serve to control and adjust cell size and to stabilize the foam structure during formation. Furthermore, it is contemplated that any one or more of the additional components described above with respect to the blowing agent compositions of the present invention could be incorporated into the foamable composition of the present invention. In such thermosetting foam embodiments, one or more of the present compositions are included as or part of a blowing agent in a foamable composition, or as a part of a two or more part foamable composition, which preferably includes one or more of the components capable of reacting and/or foaming under the proper conditions to form a foam or cellular structure.

In certain other embodiments, the one or more components comprise thermoplastic materials, particularly thermoplastic polymers and/or resins. Examples of thermoplastic foam components include polyolefins, such as for example monovinyl aromatic compounds of the formula Ar—CHCH2 wherein Ar is an aromatic hydrocarbon radical of the benzene series such as polystyrene (PS),(PS). Other examples of suitable polyolefin resins in accordance with the invention include the various ethylene resins including the ethylene homopolymers such as polyethylene (PE),and ethylene copolymers, polypropylene (PP) and polyethyleneterepthalate (PET), and foams formed there from, preferably low-density foams. In certain embodiments, the thermoplastic foamable composition is an extrudable composition.

The invention also relates to foam, and preferably closed cell foam, prepared from a polymer foam formulation containing a blowing agent comprising the compositions of the invention. In yet other embodiments, the invention provides foamable compositions comprising thermoplastic or polyolefin foams, such as polystyrene (PS), polyethylene (PE), polypropylene (PP) and polyethyleneterpthalate (PET) foams, preferably low-density foams. Any of the methods well known in the art, such as those described in “Polyurethanes Chemistry and Technology,” Volumes I and II, Saunders and Frisch, 1962, John Wiley and Sons, New York, N.Y., which is incorporated herein by reference, may be used or adapted for use in accordance with the foam embodiments of the present invention.

Other uses of the present compositions include use as solvents for example as supercritical or high pressure solvents, deposition agents, extractants, cleaning agents, and the like. Those of skill in the art will be readily able to adapt the present compositions for use in such applications without undue experimentation.

### EXAMPLES

The invention is further illustrated in the following examples which are intended to be illustrative, but not limiting in any manner.

### Example 1

The bubble (Px) and dew (Py) pressures of various mixtures of CF₃NO₂ and CO₂ are given below at 32° F. (FIG. 1A) and 100° F. (FIG. 1B), as function of CO₂ mole fraction (composition). It is observed that these pressures for any of the mixture compositions are intermediate between that of the pure components, and that they are neither above nor below those of the pure components, indicates that these compositions are non-azeotropic.

### Example 2

This example illustrates the performance characteristics of a heat transfer fluid consisting of the compositions of the present invention, which indicates that certain compositions of the present invention are excellent as replacements for each of R-507A and R404A, which are two refrigerants of known composition commonly used in low temperature and commercial refrigeration applications. The test conditions illustrate relative capacity of the compositions of the present invention based on each of the comparison refrigerants at the specific operating conditions as follows:

Mean Evaporator temp −30° F.

Mean Condenser temp 100° F.

Compressor displacement 10 ft3/min

The results are given in FIG. 2 below.

Under these conditions, it is observed that a good capacity match is obtained with R-404A and R-507A (also known as AZ-50) at 8 to 14 wt % CO2 (92 to 86 wt % HFO-1234ze) composition.

### Example 3

This example illustrates the performance characteristics of a heat transfer fluid consisting of the compositions of the present invention, which indicates that certain compositions of the present invention are excellent as replacements for each of R-410A (also known as AZ-20), R-407C and R-22, which are three refrigerants of known composition commonly used in air conditioning, heat pumps and chillers. The test conditions illustrate relative capacity of the compositions of the present invention based on each of the comparison refrigerants at the specific operating conditions as follows:

Mean Evaporator temp 35° F.

Mean Condenser temp 110° F.

Compressor displacement 10 ft3/min

The results are given in FIG. 3 below.

Under these conditions, it is observed that a good capacity match is obtained with R-22 and R-407C at 8 to 16 wt % CO2 (92 to 84 wt % CF₃NO₂) composition and a good capacity match is obtained with R-410A (also known as AZ-20) at 20 to 35 wt % CO2 (80 to 65 wt % CF₃NO₂) composition.

### Example 4A-4AM

The coefficient of performance (COP) is a universally accepted measure of refrigerant performance, especially useful in representing the relative thermodynamic efficiency of a refrigerant in a specific heating or cooling cycle involving evaporation or condensation of the refrigerant. In refrigeration engineering, this term expresses the ratio of useful refrigeration to the energy applied by the compressor in compressing the vapor. The capacity of a refrigerant represents the amount of cooling or heating it provides and provides some measure of the capability of a compressor to pump quantities of heat for a given volumetric flow rate of refrigerant. In other words, given a specific compressor, a refrigerant with a higher capacity will deliver more cooling or heating power. One means for estimating COP of a refrigerant at specific operating conditions is from the thermodynamic properties of the refrigerant using standard refrigeration cycle analysis techniques (see for example, R. C. Downing, FLUOROCARBON REFRIGERANTS HANDBOOK, Chapter 3, Prentice-Hall, 1988).

Three separate refrigeration /air conditioning cycle systems are estimated based on a specified evaporator temperature (Evap Temp), super heat, condenser temperature, sub cooling, discharge volume, and compressor efficiency for each system. The conditions for the three systems are provided in Table 4 below:

TABLE 4

| | Evap | Super | Cond | Sub | Volume | |
|---|---|---|---|---|---|---|
| Cycle | Temp, | Heat, | Temp, | Cool, | Dis., | Comp |
| Conditions | ° C. | ° C. | ° C. | ° C. | m3/s | eff |
| Air | 5 | 10 | 45 | 5 | 10 | 0.7 |
| Conditioning | | | | | | |
| Medium | −8 | 10 | 45 | 5 | 10 | 0.7 |
| Temperature | | | | | | |
| Low | −34 | 10 | 45 | 5 | 10 | 0.7 |
| Temperature | | | | | | |

The capacity and COP are determined for several compositions of the present invention over a range of relative concentrations of the components at each of the cycle conditions describe in Table 4. The results of this analysis are reported in Tables 4A-4m and FIGS. 4A1-4L4.

TABLE 4A
CF₃NO₂ AND HFO-1234YF/AIR CONDITIONING CONDITIONS

| | | | | | | Cond | | | Mass |
|---|---|---|---|---|---|---|---|---|---|
| Wt % | wt % | P_evap, | P_cond, | T_comp_exit, | Evap | Glide, | Capacity, | | Flow, |
| CF3NO2 | 1234yf | kPa | kPa | ° C. | Glide, ° C. | ° C. | kJ/s | COP | kg/s |
| 0.0% | 100.0% | 368.7 | 1135.2 | 58.9 | 0.0 | 0.0 | 23482.7 | 3.988 | 195.5 |
| 5.0% | 95.0% | 394.9 | 1214.5 | 60.2 | 1.7 | 2.0 | 25179.9 | 4.002 | 209.6 |
| 10.0% | 90.0% | 418.2 | 1286.8 | 61.2 | 2.7 | 3.3 | 26572.4 | 3.990 | 222.4 |
| 15.0% | 85.0% | 439.5 | 1353.2 | 61.8 | 3.1 | 3.9 | 27737.6 | 3.964 | 234.3 |
| 20.0% | 80.0% | 459.4 | 1414.8 | 62.2 | 3.1 | 4.0 | 28740.9 | 3.931 | 245.6 |
| 25.0% | 75.0% | 478.2 | 1471.9 | 62.5 | 2.9 | 3.7 | 29635.7 | 3.898 | 256.6 |
| 30.0% | 70.0% | 496.3 | 1524.9 | 62.6 | 2.5 | 3.3 | 30463.1 | 3.868 | 267.3 |
| 35.0% | 65.0% | 513.5 | 1573.6 | 62.6 | 2.0 | 2.7 | 31251.3 | 3.846 | 277.7 |
| 40.0% | 60.0% | 529.7 | 1617.2 | 62.5 | 1.4 | 2.0 | 32009.7 | 3.833 | 287.7 |
| 45.0% | 55.0% | 544.2 | 1654.8 | 62.4 | 0.8 | 1.3 | 32728.9 | 3.829 | 296.7 |
| 50.0% | 50.0% | 555.7 | 1685.1 | 62.4 | 0.4 | 0.7 | 33367.6 | 3.831 | 303.9 |
| 55.0% | 45.0% | 562.8 | 1706.7 | 62.5 | 0.1 | 0.3 | 33858.3 | 3.836 | 308.2 |
| 60.0% | 40.0% | 564.1 | 1718.6 | 62.9 | 0.0 | 0.0 | 34136.0 | 3.837 | 308.8 |
| 65.0% | 35.0% | 559.7 | 1720.2 | 63.6 | 0.2 | 0.0 | 34198.0 | 3.836 | 305.7 |
| 70.0% | 30.0% | 550.7 | 1711.6 | 64.3 | 0.5 | 0.2 | 34097.8 | 3.840 | 299.6 |
| 75.0% | 25.0% | 538.5 | 1693.4 | 65.2 | 0.9 | 0.4 | 33893.3 | 3.853 | 291.7 |
| 80.0% | 20.0% | 523.9 | 1666.2 | 65.9 | 1.3 | 0.7 | 33608.7 | 3.878 | 282.5 |
| 85.0% | 15.0% | 507.4 | 1630.5 | 66.6 | 1.5 | 1.0 | 33229.8 | 3.913 | 272.4 |
| 90.0% | 10.0% | 488.7 | 1586.3 | 67.1 | 1.5 | 1.0 | 32704.0 | 3.953 | 261.3 |
| 95.0% | 5.0% | 467.0 | 1533.0 | 67.5 | 1.1 | 0.8 | 31935.1 | 3.989 | 248.8 |
| 100.0% | 0.0% | 441.0 | 1469.0 | 67.6 | 0.0 | 0.0 | 30771.3 | 4.007 | 234.3 |

TABLE 4B
CF₃NO₂ AND HFO-1234YF/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | 1234yf | kPa | kPa | ° C. | Glide, ° C. | Glide, ° C. | kJ/s | COP | kg/s |
| 0.0% | 100.0% | 236.5 | 1135.2 | 61.3 | 0.0 | 0.0 | 14173.6 | 2.655 | 127.5 |
| 5.0% | 95.0% | 253.3 | 1214.5 | 62.6 | 1.5 | 2.0 | 15197.2 | 2.664 | 136.6 |
| 10.0% | 90.0% | 268.2 | 1286.8 | 63.6 | 2.4 | 3.3 | 16028.9 | 2.655 | 144.9 |
| 15.0% | 85.0% | 281.9 | 1353.2 | 64.4 | 2.7 | 3.9 | 16722.0 | 2.636 | 152.5 |
| 20.0% | 80.0% | 294.7 | 1414.8 | 64.9 | 2.8 | 4.0 | 17320.4 | 2.613 | 159.8 |
| 25.0% | 75.0% | 306.9 | 1471.9 | 65.2 | 2.5 | 3.7 | 17859.9 | 2.590 | 166.9 |
| 30.0% | 70.0% | 318.8 | 1524.9 | 65.4 | 2.2 | 3.3 | 18367.9 | 2.570 | 173.9 |
| 35.0% | 65.0% | 330.3 | 1573.6 | 65.5 | 1.7 | 2.7 | 18863.9 | 2.555 | 180.8 |
| 40.0% | 60.0% | 341.4 | 1617.2 | 65.5 | 1.2 | 2.0 | 19353.3 | 2.546 | 187.5 |
| 45.0% | 55.0% | 351.3 | 1654.8 | 65.5 | 0.7 | 1.3 | 19827.9 | 2.544 | 193.6 |
| 50.0% | 50.0% | 359.2 | 1685.1 | 65.5 | 0.2 | 0.7 | 20251.0 | 2.548 | 198.5 |
| 55.0% | 45.0% | 363.6 | 1706.7 | 65.8 | 0.0 | 0.3 | 20552.6 | 2.551 | 201.1 |
| 60.0% | 40.0% | 363.1 | 1718.6 | 66.4 | 0.0 | 0.0 | 20670.9 | 2.551 | 200.7 |
| 65.0% | 35.0% | 358.1 | 1720.2 | 67.4 | 0.3 | 0.0 | 20623.0 | 2.549 | 197.5 |
| 70.0% | 30.0% | 350.2 | 1711.6 | 68.5 | 0.7 | 0.2 | 20481.6 | 2.552 | 192.5 |
| 75.0% | 25.0% | 340.6 | 1693.4 | 69.6 | 1.1 | 0.4 | 20299.6 | 2.565 | 186.5 |
| 80.0% | 20.0% | 330.0 | 1666.2 | 70.7 | 1.4 | 0.7 | 20092.2 | 2.586 | 180.0 |
| 85.0% | 15.0% | 318.4 | 1630.5 | 71.6 | 1.7 | 1.0 | 19844.4 | 2.615 | 173.1 |
| 90.0% | 10.0% | 305.6 | 1586.3 | 72.3 | 1.6 | 1.0 | 19514.4 | 2.648 | 165.7 |
| 95.0% | 5.0% | 290.8 | 1533.0 | 72.9 | 1.2 | 0.8 | 19028.6 | 2.678 | 157.4 |
| 100.0% | 0.0% | 272.9 | 1469.0 | 73.3 | 0.0 | 0.0 | 18270.3 | 2.695 | 147.5 |

TABLE 4C
CF₃NO₂ AND HFO-1234YF/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | 1234yf | kPa | kPa | ° C. | Glide, ° C. | Glide, ° C. | kJ/s | COP | kg/s |
| 0.0% | 100.0% | 82.6 | 1135.2 | 69.1 | 0.0 | 0.0 | 4376.8 | 1.340 | 47.2 |
| 5.0% | 95.0% | 88.5 | 1214.5 | 70.5 | 1.2 | 2.0 | 4695.1 | 1.344 | 50.5 |
| 10.0% | 90.0% | 93.7 | 1286.8 | 71.6 | 1.9 | 3.3 | 4948.6 | 1.338 | 53.5 |
| 15.0% | 85.0% | 98.5 | 1353.2 | 72.5 | 2.1 | 3.9 | 5158.0 | 1.327 | 56.3 |
| 20.0% | 80.0% | 103.0 | 1414.8 | 73.2 | 2.1 | 4.0 | 5340.1 | 1.314 | 59.0 |
| 25.0% | 75.0% | 107.5 | 1471.9 | 73.7 | 1.9 | 3.7 | 5508.3 | 1.301 | 61.6 |
| 30.0% | 70.0% | 111.9 | 1524.9 | 74.0 | 1.5 | 3.3 | 5673.1 | 1.290 | 64.3 |
| 35.0% | 65.0% | 116.3 | 1573.6 | 74.3 | 1.2 | 2.7 | 5842.1 | 1.282 | 67.0 |
| 40.0% | 60.0% | 120.7 | 1617.2 | 74.4 | 0.8 | 2.0 | 6018.2 | 1.277 | 69.7 |
| 45.0% | 55.0% | 124.8 | 1654.8 | 74.5 | 0.4 | 1.3 | 6197.3 | 1.277 | 72.3 |
| 50.0% | 50.0% | 128.0 | 1685.1 | 74.7 | 0.1 | 0.7 | 6355.5 | 1.280 | 74.3 |
| 55.0% | 45.0% | 128.8 | 1706.7 | 75.5 | 0.0 | 0.3 | 6425.6 | 1.281 | 74.7 |
| 60.0% | 40.0% | 126.3 | 1718.6 | 77.1 | 0.2 | 0.0 | 6366.3 | 1.277 | 73.2 |
| 65.0% | 35.0% | 122.3 | 1720.2 | 79.0 | 0.6 | 0.0 | 6261.3 | 1.275 | 70.7 |
| 70.0% | 30.0% | 117.8 | 1711.6 | 80.9 | 1.0 | 0.2 | 6159.2 | 1.280 | 68.0 |
| 75.0% | 25.0% | 113.4 | 1693.4 | 82.6 | 1.4 | 0.4 | 6071.1 | 1.292 | 65.2 |
| 80.0% | 20.0% | 108.9 | 1666.2 | 84.2 | 1.8 | 0.7 | 5994.1 | 1.311 | 62.5 |
| 85.0% | 15.0% | 104.4 | 1630.5 | 85.6 | 2.0 | 1.0 | 5915.8 | 1.334 | 59.8 |
| 90.0% | 10.0% | 99.5 | 1586.3 | 86.9 | 1.9 | 1.0 | 5814.6 | 1.359 | 57.0 |
| 95.0% | 5.0% | 93.9 | 1533.0 | 88.0 | 1.4 | 0.8 | 5655.0 | 1.382 | 53.8 |
| 100.0% | 0.0% | 86.8 | 1469.0 | 89.1 | 0.0 | 0.0 | 5375.7 | 1.396 | 49.7 |

TABLE 4D
CF₃NO₂ AND HFO-1234ZE(E)/AIR CONDITIONING CONDITIONS

| | | | | | | | | | Mass |
|---|---|---|---|---|---|---|---|---|---|
| Wt % | Wt % | P_evap, | P_cond, | T_comp_exit, | Evap | Cond | Capacity, | | Flow, |
| CF3NO2 | 1234zeE | kPa | kPa | ° C. | Glide, ° C. | Glide, ° C. | kJ/s | COP | kg/s |
| 0.0% | 100.0% | 252.1 | 863.1 | 61.7 | 0.0 | 0.0 | 18485.8 | 4.125 | 129.9 |
| 5.0% | 95.0% | 268.5 | 913.6 | 62.6 | 1.4 | 1.5 | 19634.6 | 4.138 | 138.4 |
| 10.0% | 90.0% | 283.9 | 961.6 | 63.4 | 2.3 | 2.6 | 20659.3 | 4.136 | 146.5 |
| 15.0% | 85.0% | 298.5 | 1007.6 | 63.9 | 2.9 | 3.2 | 21585.6 | 4.124 | 154.3 |
| 20.0% | 80.0% | 312.7 | 1051.9 | 64.3 | 3.3 | 3.6 | 22439.4 | 4.107 | 162.0 |
| 25.0% | 75.0% | 326.5 | 1094.7 | 64.6 | 3.4 | 3.8 | 23240.0 | 4.086 | 169.5 |
| 30.0% | 70.0% | 340.1 | 1136.4 | 64.8 | 3.3 | 3.7 | 24004.0 | 4.065 | 177.0 |
| 35.0% | 65.0% | 353.6 | 1176.8 | 65.0 | 3.1 | 3.5 | 24744.0 | 4.046 | 184.6 |
| 40.0% | 60.0% | 367.0 | 1215.9 | 65.0 | 2.8 | 3.2 | 25469.0 | 4.029 | 192.2 |
| 45.0% | 55.0% | 380.2 | 1253.7 | 65.0 | 2.4 | 2.8 | 26183.5 | 4.015 | 199.8 |
| 50.0% | 50.0% | 393.2 | 1289.8 | 64.9 | 2.0 | 2.3 | 26887.8 | 4.006 | 207.3 |
| 55.0% | 45.0% | 405.6 | 1323.8 | 64.9 | 1.5 | 1.9 | 27574.3 | 4.000 | 214.5 |
| 60.0% | 40.0% | 417.2 | 1355.4 | 64.8 | 1.1 | 1.4 | 28232.1 | 3.998 | 221.3 |
| 65.0% | 35.0% | 427.6 | 1384.0 | 64.8 | 0.7 | 1.0 | 28844.3 | 3.998 | 227.4 |
| 70.0% | 30.0% | 436.4 | 1409.3 | 64.9 | 0.4 | 0.7 | 29392.0 | 3.998 | 232.5 |
| 75.0% | 25.0% | 443.1 | 1430.7 | 65.1 | 0.2 | 0.4 | 29859.0 | 3.999 | 236.4 |
| 80.0% | 20.0% | 447.5 | 1447.9 | 65.4 | 0.1 | 0.2 | 30232.7 | 3.999 | 238.9 |
| 85.0% | 15.0% | 449.5 | 1460.6 | 65.8 | 0.0 | 0.1 | 30509.6 | 3.999 | 239.9 |
| 90.0% | 10.0% | 449.1 | 1468.6 | 66.4 | 0.0 | 0.0 | 30691.5 | 4.000 | 239.5 |
| 95.0% | 5.0% | 446.3 | 1471.5 | 67.0 | 0.0 | 0.0 | 30780.7 | 4.002 | 237.6 |
| 100.0% | 0.0% | 441.0 | 1469.0 | 67.6 | 0.0 | 0.0 | 30771.3 | 4.007 | 234.3 |

TABLE 4E
CF₃NO₂ AND HFO-1234ZE E(E)/MEDIUM TEMPERATURE CONDITIONS

| | | | | | | | | | Mass |
|---|---|---|---|---|---|---|---|---|---|
| Wt % | Wt % | P_evap, | P_cond, | T_comp_exit, | Evap | Cond | Capacity, | | Flow, |
| CF3NO2 | 1234zeE | kPa | kPa | ° C. | Glide, ° C. | Glide, ° C. | kJ/s | COP | kg/s |
| 0.0% | 100.0% | 154.5 | 863.1 | 65.4 | 0.0 | 0.0 | 10836.1 | 2.774 | 81.5 |
| 5.0% | 95.0% | 165.0 | 913.6 | 66.3 | 1.3 | 1.5 | 11535.0 | 2.783 | 87.0 |
| 10.0% | 90.0% | 174.7 | 961.6 | 67.1 | 2.2 | 2.6 | 12154.3 | 2.781 | 92.2 |
| 15.0% | 85.0% | 184.0 | 1007.6 | 67.7 | 2.8 | 3.2 | 12713.5 | 2.772 | 97.2 |
| 20.0% | 80.0% | 193.0 | 1051.9 | 68.2 | 3.1 | 3.6 | 13229.0 | 2.759 | 102.1 |
| 25.0% | 75.0% | 201.8 | 1094.7 | 68.5 | 3.2 | 3.8 | 13714.5 | 2.744 | 106.9 |
| 30.0% | 70.0% | 210.6 | 1136.4 | 68.8 | 3.1 | 3.7 | 14181.3 | 2.729 | 111.8 |
| 35.0% | 65.0% | 219.3 | 1176.8 | 69.0 | 2.9 | 3.5 | 14638.4 | 2.715 | 116.7 |
| 40.0% | 60.0% | 228.1 | 1215.9 | 69.1 | 2.6 | 3.2 | 15092.2 | 2.703 | 121.7 |
| 45.0% | 55.0% | 236.8 | 1253.7 | 69.1 | 2.2 | 2.8 | 15546.1 | 2.694 | 126.7 |
| 50.0% | 50.0% | 245.5 | 1289.8 | 69.1 | 1.8 | 2.3 | 16000.6 | 2.688 | 131.8 |
| 55.0% | 45.0% | 253.9 | 1323.8 | 69.0 | 1.4 | 1.9 | 16449.1 | 2.685 | 136.7 |
| 60.0% | 40.0% | 261.8 | 1355.4 | 69.0 | 1.0 | 1.4 | 16882.3 | 2.684 | 141.3 |
| 65.0% | 35.0% | 268.9 | 1384.0 | 69.1 | 0.6 | 1.0 | 17284.6 | 2.685 | 145.4 |
| 70.0% | 30.0% | 274.7 | 1409.3 | 69.2 | 0.3 | 0.7 | 17638.2 | 2.687 | 148.8 |
| 75.0% | 25.0% | 278.9 | 1430.7 | 69.5 | 0.1 | 0.4 | 17925.9 | 2.688 | 151.3 |
| 80.0% | 20.0% | 281.3 | 1447.9 | 70.0 | 0.0 | 0.2 | 18138.0 | 2.688 | 152.6 |
| 85.0% | 15.0% | 281.8 | 1460.6 | 70.7 | 0.0 | 0.1 | 18274.2 | 2.688 | 152.9 |
| 90.0% | 10.0% | 280.5 | 1468.6 | 71.5 | 0.0 | 0.0 | 18339.8 | 2.689 | 152.0 |
| 95.0% | 5.0% | 277.6 | 1471.5 | 72.3 | 0.1 | 0.0 | 18339.9 | 2.691 | 150.2 |
| 100.0% | 0.0% | 272.9 | 1469.0 | 73.3 | 0.0 | 0.0 | 18270.3 | 2.695 | 147.5 |

TABLE 4F
CF₃NO₂ AND HFO-1234ZE(E)/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | 1234zeE | kPa | kPa | ° C. | Glide, ° C. | Glide, ° C. | kJ/s | COP | kg/s |
| 0.0% | 100.0% | 48.2 | 863.1 | 76.6 | 0.0 | 0.0 | 3100.5 | 1.436 | 27.2 |
| 5.0% | 95.0% | 51.8 | 913.6 | 77.5 | 1.2 | 1.5 | 3321.1 | 1.441 | 29.2 |
| 10.0% | 90.0% | 55.1 | 961.6 | 78.3 | 2.0 | 2.6 | 3513.6 | 1.439 | 31.1 |
| 15.0% | 85.0% | 58.3 | 1007.6 | 79.0 | 2.5 | 3.2 | 3685.8 | 1.433 | 32.8 |
| 20.0% | 80.0% | 61.3 | 1051.9 | 79.6 | 2.7 | 3.6 | 3844.6 | 1.425 | 34.6 |
| 25.0% | 75.0% | 64.3 | 1094.7 | 80.1 | 2.8 | 3.8 | 3995.3 | 1.416 | 36.3 |
| 30.0% | 70.0% | 67.4 | 1136.4 | 80.5 | 2.7 | 3.7 | 4142.6 | 1.407 | 38.1 |
| 35.0% | 65.0% | 70.4 | 1176.8 | 80.7 | 2.5 | 3.5 | 4290.4 | 1.399 | 39.9 |
| 40.0% | 60.0% | 73.6 | 1215.9 | 80.9 | 2.2 | 3.2 | 4441.7 | 1.392 | 41.8 |
| 45.0% | 55.0% | 76.9 | 1253.7 | 81.0 | 1.9 | 2.8 | 4598.4 | 1.387 | 43.7 |
| 50.0% | 50.0% | 80.2 | 1289.8 | 81.0 | 1.5 | 2.3 | 4761.3 | 1.384 | 45.7 |
| 55.0% | 45.0% | 83.5 | 1323.8 | 81.0 | 1.1 | 1.9 | 4928.1 | 1.383 | 47.7 |
| 60.0% | 40.0% | 86.7 | 1355.4 | 81.0 | 0.8 | 1.4 | 5093.7 | 1.384 | 49.6 |
| 65.0% | 35.0% | 89.6 | 1384.0 | 81.1 | 0.4 | 1.0 | 5247.6 | 1.386 | 51.4 |
| 70.0% | 30.0% | 91.8 | 1409.3 | 81.4 | 0.2 | 0.7 | 5375.7 | 1.388 | 52.7 |
| 75.0% | 25.0% | 93.1 | 1430.7 | 82.1 | 0.0 | 0.4 | 5464.7 | 1.389 | 53.5 |
| 80.0% | 20.0% | 93.4 | 1447.9 | 83.1 | 0.0 | 0.2 | 5509.0 | 1.389 | 53.7 |
| 85.0% | 15.0% | 92.7 | 1460.6 | 84.3 | 0.1 | 0.1 | 5514.9 | 1.389 | 53.3 |
| 90.0% | 10.0% | 91.4 | 1468.6 | 85.8 | 0.1 | 0.0 | 5492.4 | 1.390 | 52.5 |
| 95.0% | 5.0% | 89.4 | 1471.5 | 87.4 | 0.2 | 0.0 | 5447.6 | 1.392 | 51.3 |
| 100.0% | 0.0% | 86.8 | 1469.0 | 89.1 | 0.0 | 0.0 | 5375.7 | 1.396 | 49.7 |

TABLE 4G
CF₃NO₂ AND PROPANE/AIR CONDITIONING CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | PROPANE | kPa | kPa | ° C. | Glide, ° C. | Glide, ° C. | kJ/s | COP | kg/s |
| 0.0% | 100.0% | 551.3 | 1534.4 | 67.8 | 0.0 | 0.0 | 33037.3 | 4.029 | 113.7 |
| 5.0% | 95.0% | 547.9 | 1529.9 | 67.8 | 0.0 | 0.0 | 32934.0 | 4.029 | 116.5 |
| 10.0% | 90.0% | 544.4 | 1525.3 | 67.8 | 0.1 | 0.0 | 32827.3 | 4.028 | 119.5 |
| 15.0% | 85.0% | 540.8 | 1520.6 | 67.8 | 0.1 | 0.0 | 32717.2 | 4.028 | 122.7 |
| 20.0% | 80.0% | 537.0 | 1515.7 | 67.8 | 0.1 | 0.0 | 32603.6 | 4.028 | 126.1 |
| 25.0% | 75.0% | 533.0 | 1510.7 | 67.7 | 0.2 | 0.0 | 32486.4 | 4.028 | 129.7 |
| 30.0% | 70.0% | 528.9 | 1505.6 | 67.7 | 0.2 | 0.1 | 32365.6 | 4.028 | 133.5 |
| 35.0% | 65.0% | 524.7 | 1500.4 | 67.6 | 0.2 | 0.1 | 32241.4 | 4.028 | 137.5 |
| 40.0% | 60.0% | 520.2 | 1495.1 | 67.6 | 0.2 | 0.1 | 32113.7 | 4.027 | 141.9 |
| 45.0% | 55.0% | 515.5 | 1489.8 | 67.6 | 0.2 | 0.1 | 31983.0 | 4.027 | 146.5 |
| 50.0% | 50.0% | 510.6 | 1484.5 | 67.5 | 0.2 | 0.0 | 31849.7 | 4.027 | 151.6 |
| 55.0% | 45.0% | 505.5 | 1479.3 | 67.4 | 0.2 | 0.0 | 31714.4 | 4.026 | 157.0 |
| 60.0% | 40.0% | 500.2 | 1474.3 | 67.4 | 0.2 | 0.0 | 31578.2 | 4.026 | 162.8 |
| 65.0% | 35.0% | 494.6 | 1469.6 | 67.3 | 0.2 | 0.0 | 31442.6 | 4.025 | 169.2 |
| 70.0% | 30.0% | 488.7 | 1465.4 | 67.3 | 0.2 | 0.0 | 31309.6 | 4.024 | 176.2 |
| 75.0% | 25.0% | 482.5 | 1462.0 | 67.2 | 0.2 | 0.0 | 31182.3 | 4.022 | 183.8 |
| 80.0% | 20.0% | 475.9 | 1459.6 | 67.2 | 0.2 | 0.0 | 31064.2 | 4.020 | 192.2 |
| 85.0% | 15.0% | 468.8 | 1458.6 | 67.2 | 0.1 | 0.0 | 30961.1 | 4.017 | 201.5 |
| 90.0% | 10.0% | 461.0 | 1459.6 | 67.2 | 0.1 | 0.0 | 30877.0 | 4.014 | 211.7 |
| 95.0% | 5.0% | 452.1 | 1463.0 | 67.4 | 0.1 | 0.0 | 30816.3 | 4.010 | 222.8 |
| 100.0% | 0.0% | 441.0 | 1469.0 | 67.6 | 0.0 | 0.0 | 30771.3 | 4.007 | 234.3 |

TABLE 4H
CF₃NO₂ AND PROPANE/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | PROPANE | kPa | kPa | ° C. | Glide, ° C. | Glide, ° C. | kJ/s | COP | kg/s |
| 0.0% | 100.0% | 368.8 | 1534.4 | 73.7 | 0.0 | 0.0 | 21279.7 | 2.730 | 77.4 |
| 5.0% | 95.0% | 366.0 | 1529.9 | 73.7 | 0.0 | 0.0 | 21179.8 | 2.729 | 79.2 |
| 10.0% | 90.0% | 363.0 | 1525.3 | 73.7 | 0.1 | 0.0 | 21076.3 | 2.729 | 81.1 |
| 15.0% | 85.0% | 360.0 | 1520.6 | 73.7 | 0.1 | 0.0 | 20969.0 | 2.728 | 83.1 |
| 20.0% | 80.0% | 356.9 | 1515.7 | 73.6 | 0.2 | 0.0 | 20857.7 | 2.727 | 85.2 |
| 25.0% | 75.0% | 353.6 | 1510.7 | 73.6 | 0.2 | 0.0 | 20742.2 | 2.726 | 87.5 |
| 30.0% | 70.0% | 350.2 | 1505.6 | 73.5 | 0.2 | 0.1 | 20622.1 | 2.725 | 89.9 |
| 35.0% | 65.0% | 346.6 | 1500.4 | 73.5 | 0.3 | 0.1 | 20497.2 | 2.725 | 92.4 |
| 40.0% | 60.0% | 342.9 | 1495.1 | 73.4 | 0.3 | 0.1 | 20367.3 | 2.724 | 95.2 |
| 45.0% | 55.0% | 339.0 | 1489.8 | 73.4 | 0.3 | 0.1 | 20232.0 | 2.723 | 98.0 |
| 50.0% | 50.0% | 334.9 | 1484.5 | 73.3 | 0.3 | 0.0 | 20091.2 | 2.722 | 101.1 |
| 55.0% | 45.0% | 330.6 | 1479.3 | 73.2 | 0.3 | 0.0 | 19944.5 | 2.720 | 104.4 |
| 60.0% | 40.0% | 326.1 | 1474.3 | 73.1 | 0.3 | 0.0 | 19791.8 | 2.719 | 108.0 |
| 65.0% | 35.0% | 321.3 | 1469.6 | 73.0 | 0.3 | 0.0 | 19632.7 | 2.717 | 111.8 |
| 70.0% | 30.0% | 316.2 | 1465.4 | 72.9 | 0.3 | 0.0 | 19467.2 | 2.715 | 116.0 |
| 75.0% | 25.0% | 310.8 | 1462.0 | 72.9 | 0.3 | 0.0 | 19295.1 | 2.713 | 120.5 |
| 80.0% | 20.0% | 305.0 | 1459.6 | 72.8 | 0.3 | 0.0 | 19115.8 | 2.710 | 125.3 |
| 85.0% | 15.0% | 298.6 | 1458.6 | 72.8 | 0.3 | 0.0 | 18929.0 | 2.707 | 130.5 |
| 90.0% | 10.0% | 291.4 | 1459.6 | 72.8 | 0.2 | 0.0 | 18731.8 | 2.703 | 136.1 |
| 95.0% | 5.0% | 283.2 | 1463.0 | 73.0 | 0.1 | 0.0 | 18518.5 | 2.699 | 141.9 |
| 100.0% | 0.0% | 272.9 | 1469.0 | 73.3 | 0.0 | 0.0 | 18270.3 | 2.695 | 147.5 |

TABLE 4I
CF₃NO₂ AND PROPANE/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | PROPANE | kPa | kPa | ° C. | Glide, ° C. | Glide, ° C. | kJ/s | COP | kg/s |
| 0.0% | 100.0% | 142.9 | 1534.4 | 120.1 | 0.0 | 0.0 | 7710.2 | 1.030 | 31.7 |
| 5.0% | 95.0% | 141.2 | 1529.9 | 89.9 | 0.1 | 0.0 | 7640.4 | 1.440 | 32.3 |
| 10.0% | 90.0% | 139.5 | 1525.3 | 89.9 | 0.1 | 0.0 | 7568.2 | 1.439 | 33.0 |
| 15.0% | 85.0% | 137.7 | 1520.6 | 89.9 | 0.2 | 0.0 | 7493.6 | 1.437 | 33.7 |
| 20.0% | 80.0% | 135.8 | 1515.7 | 89.9 | 0.3 | 0.0 | 7416.4 | 1.436 | 34.4 |
| 25.0% | 75.0% | 133.9 | 1510.7 | 89.8 | 0.3 | 0.0 | 7336.2 | 1.435 | 35.1 |
| 30.0% | 70.0% | 131.9 | 1505.6 | 89.8 | 0.4 | 0.1 | 7252.8 | 1.433 | 35.9 |
| 35.0% | 65.0% | 129.9 | 1500.4 | 89.7 | 0.4 | 0.1 | 7165.8 | 1.432 | 36.7 |
| 40.0% | 60.0% | 127.7 | 1495.1 | 89.6 | 0.5 | 0.1 | 7075.0 | 1.430 | 37.5 |
| 45.0% | 55.0% | 125.4 | 1489.8 | 89.5 | 0.5 | 0.1 | 6979.9 | 1.428 | 38.4 |
| 50.0% | 50.0% | 123.1 | 1484.5 | 89.4 | 0.5 | 0.0 | 6880.0 | 1.426 | 39.4 |
| 55.0% | 45.0% | 120.6 | 1479.3 | 89.3 | 0.6 | 0.0 | 6774.7 | 1.424 | 40.3 |
| 60.0% | 40.0% | 118.0 | 1474.3 | 89.2 | 0.6 | 0.0 | 6663.4 | 1.422 | 41.4 |
| 65.0% | 35.0% | 115.2 | 1469.6 | 89.0 | 0.6 | 0.0 | 6545.2 | 1.420 | 42.5 |
| 70.0% | 30.0% | 112.2 | 1465.4 | 88.9 | 0.6 | 0.0 | 6419.1 | 1.417 | 43.6 |
| 75.0% | 25.0% | 109.0 | 1462.0 | 88.8 | 0.6 | 0.0 | 6283.9 | 1.414 | 44.7 |
| 80.0% | 20.0% | 105.5 | 1459.6 | 88.7 | 0.6 | 0.0 | 6137.9 | 1.410 | 45.9 |
| 85.0% | 15.0% | 101.7 | 1458.6 | 88.6 | 0.5 | 0.0 | 5978.9 | 1.407 | 47.1 |
| 90.0% | 10.0% | 97.5 | 1459.6 | 88.6 | 0.5 | 0.0 | 5803.7 | 1.403 | 48.2 |
| 95.0% | 5.0% | 92.6 | 1463.0 | 88.8 | 0.3 | 0.0 | 5607.0 | 1.399 | 49.2 |
| 100.0% | 0.0% | 86.8 | 1469.0 | 89.1 | 0.0 | 0.0 | 5375.7 | 1.396 | 49.7 |

TABLE 4J
CF₃NO₂ AND ISOBUTANE/AIR CONDITIONING TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Isobutane | kPa | kPa | ° C. | Glide, ° C. | Glide, ° C. | kJ/s | COP | kg/s |
| 0.0% | 100.0% | 186.4 | 604.2 | 60.2 | 0.0 | 0.0 | 13489.7 | 4.220 | 47.9 |
| 5.0% | 95.0% | 189.5 | 615.3 | 60.5 | 0.2 | 0.4 | 13738.2 | 4.221 | 50.0 |
| 10.0% | 90.0% | 192.9 | 627.3 | 60.8 | 0.5 | 0.8 | 14007.8 | 4.223 | 52.2 |
| 15.0% | 85.0% | 196.6 | 640.4 | 61.2 | 0.7 | 1.2 | 14300.8 | 4.224 | 54.6 |
| 20.0% | 80.0% | 200.6 | 654.8 | 61.5 | 1.0 | 1.6 | 14619.7 | 4.226 | 57.3 |
| 25.0% | 75.0% | 205.0 | 670.5 | 61.9 | 1.3 | 2.1 | 14969.0 | 4.227 | 60.2 |
| 30.0% | 70.0% | 209.9 | 687.7 | 62.3 | 1.5 | 2.5 | 15351.1 | 4.228 | 63.5 |
| 35.0% | 65.0% | 215.3 | 706.8 | 62.8 | 1.8 | 3.0 | 15771.1 | 4.228 | 67.1 |
| 40.0% | 60.0% | 221.3 | 727.9 | 63.2 | 2.1 | 3.5 | 16234.0 | 4.228 | 71.1 |
| 45.0% | 55.0% | 228.0 | 751.5 | 63.7 | 2.4 | 3.9 | 16746.0 | 4.227 | 75.7 |
| 50.0% | 50.0% | 235.4 | 777.8 | 64.2 | 2.7 | 4.4 | 17313.9 | 4.224 | 80.8 |
| 55.0% | 45.0% | 243.9 | 807.5 | 64.7 | 3.0 | 4.8 | 17947.6 | 4.220 | 86.6 |
| 60.0% | 40.0% | 253.5 | 841.1 | 65.2 | 3.3 | 5.2 | 18657.9 | 4.214 | 93.3 |
| 65.0% | 35.0% | 264.4 | 879.5 | 65.7 | 3.5 | 5.5 | 19458.2 | 4.206 | 101.0 |
| 70.0% | 30.0% | 277.1 | 923.8 | 66.3 | 3.7 | 5.8 | 20366.6 | 4.194 | 110.0 |
| 75.0% | 25.0% | 291.9 | 975.5 | 66.8 | 3.7 | 5.9 | 21407.1 | 4.178 | 120.7 |
| 80.0% | 20.0% | 309.5 | 1036.5 | 67.3 | 3.6 | 5.7 | 22613.2 | 4.157 | 133.6 |
| 85.0% | 15.0% | 330.8 | 1110.0 | 67.7 | 3.3 | 5.3 | 24034.9 | 4.129 | 149.5 |
| 90.0% | 10.0% | 357.6 | 1200.5 | 67.9 | 2.7 | 4.4 | 25752.8 | 4.095 | 169.7 |
| 95.0% | 5.0% | 392.4 | 1315.6 | 68.0 | 1.7 | 2.7 | 27909.7 | 4.054 | 196.5 |
| 100.0% | 0.0% | 441.0 | 1469.0 | 67.6 | 0.0 | 0.0 | 30771.3 | 4.007 | 234.3 |

TABLE 4K
CF₃NO₂ AND ISOBUTANE/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Isobutane | kPa | kPa | ° C. | Glide, ° C. | Glide, ° C. | kJ/s | COP | kg/s |
| 0.0% | 100.0% | 116.7 | 604.2 | 63.9 | 0.0 | 0.0 | 8136.5 | 2.852 | 30.9 |
| 5.0% | 95.0% | 118.6 | 615.3 | 64.2 | 0.2 | 0.4 | 8282.4 | 2.854 | 32.2 |
| 10.0% | 90.0% | 120.7 | 627.3 | 64.6 | 0.4 | 0.8 | 8440.5 | 2.855 | 33.6 |
| 15.0% | 85.0% | 122.9 | 640.4 | 65.0 | 0.6 | 1.2 | 8612.4 | 2.857 | 35.2 |
| 20.0% | 80.0% | 125.3 | 654.8 | 65.4 | 0.8 | 1.6 | 8799.7 | 2.858 | 36.8 |
| 25.0% | 75.0% | 128.0 | 670.5 | 65.8 | 1.0 | 2.1 | 9004.5 | 2.859 | 38.7 |
| 30.0% | 70.0% | 130.9 | 687.7 | 66.2 | 1.3 | 2.5 | 9228.4 | 2.860 | 40.8 |
| 35.0% | 65.0% | 134.2 | 706.8 | 66.7 | 1.5 | 3.0 | 9474.6 | 2.860 | 43.0 |
| 40.0% | 60.0% | 137.8 | 727.9 | 67.2 | 1.8 | 3.5 | 9746.0 | 2.860 | 45.6 |
| 45.0% | 55.0% | 141.9 | 751.5 | 67.8 | 2.0 | 3.9 | 10046.1 | 2.859 | 48.4 |
| 50.0% | 50.0% | 146.4 | 777.8 | 68.3 | 2.3 | 4.4 | 10379.3 | 2.858 | 51.6 |
| 55.0% | 45.0% | 151.6 | 807.5 | 68.9 | 2.5 | 4.8 | 10750.2 | 2.855 | 55.3 |
| 60.0% | 40.0% | 157.4 | 841.1 | 69.5 | 2.7 | 5.2 | 11166.0 | 2.851 | 59.5 |
| 65.0% | 35.0% | 164.0 | 879.5 | 70.1 | 2.9 | 5.5 | 11634.5 | 2.845 | 64.3 |
| 70.0% | 30.0% | 171.8 | 923.8 | 70.8 | 3.0 | 5.8 | 12166.0 | 2.837 | 70.0 |
| 75.0% | 25.0% | 180.8 | 975.5 | 71.4 | 3.1 | 5.9 | 12774.6 | 2.825 | 76.7 |
| 80.0% | 20.0% | 191.6 | 1036.5 | 72.0 | 3.0 | 5.7 | 13480.2 | 2.810 | 84.8 |
| 85.0% | 15.0% | 204.7 | 1110.0 | 72.6 | 2.7 | 5.3 | 14312.6 | 2.790 | 94.7 |
| 90.0% | 10.0% | 221.1 | 1200.5 | 73.1 | 2.2 | 4.4 | 15319.9 | 2.765 | 107.3 |
| 95.0% | 5.0% | 242.7 | 1315.6 | 73.3 | 1.4 | 2.7 | 16588.4 | 2.734 | 124.0 |
| 100.0% | 0.0% | 272.9 | 1469.0 | 73.3 | 0.0 | 0.0 | 18270.3 | 2.695 | 147.5 |

TABLE 4L
CF₃NO₂ AND ISOBUTANE/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Isobutane | kPa | kPa | ° C. | Glide, ° C. | Glide, ° C. | kJ/s | COP | kg/s |
| 0.0% | 100.0% | 38.4 | 604.2 | 75.6 | 0.0 | 0.0 | 2493.4 | 1.498 | 11.0 |
| 5.0% | 95.0% | 38.9 | 615.3 | 76.0 | 0.1 | 0.4 | 2535.8 | 1.499 | 11.4 |
| 10.0% | 90.0% | 39.6 | 627.3 | 76.4 | 0.2 | 0.8 | 2581.8 | 1.500 | 11.9 |
| 15.0% | 85.0% | 40.2 | 640.4 | 76.9 | 0.4 | 1.2 | 2631.7 | 1.502 | 12.4 |
| 20.0% | 80.0% | 41.0 | 654.8 | 77.4 | 0.5 | 1.6 | 2686.0 | 1.503 | 13.0 |
| 25.0% | 75.0% | 41.8 | 670.5 | 77.9 | 0.6 | 2.1 | 2745.2 | 1.503 | 13.6 |
| 30.0% | 70.0% | 42.7 | 687.7 | 78.5 | 0.8 | 2.5 | 2810.0 | 1.504 | 14.3 |
| 35.0% | 65.0% | 43.7 | 706.8 | 79.0 | 0.9 | 3.0 | 2881.1 | 1.505 | 15.1 |
| 40.0% | 60.0% | 44.8 | 727.9 | 79.6 | 1.1 | 3.5 | 2959.4 | 1.505 | 16.0 |
| 45.0% | 55.0% | 46.0 | 751.5 | 80.3 | 1.3 | 3.9 | 3045.8 | 1.505 | 16.9 |
| 50.0% | 50.0% | 47.4 | 777.8 | 81.0 | 1.4 | 4.4 | 3141.7 | 1.504 | 18.0 |
| 55.0% | 45.0% | 49.0 | 807.5 | 81.7 | 1.6 | 4.8 | 3248.4 | 1.502 | 19.2 |
| 60.0% | 40.0% | 50.8 | 841.1 | 82.5 | 1.7 | 5.2 | 3367.8 | 1.500 | 20.7 |
| 65.0% | 35.0% | 52.9 | 879.5 | 83.3 | 1.8 | 5.5 | 3502.1 | 1.497 | 22.3 |
| 70.0% | 30.0% | 55.2 | 923.8 | 84.1 | 1.9 | 5.8 | 3654.3 | 1.492 | 24.2 |
| 75.0% | 25.0% | 58.0 | 975.5 | 85.0 | 1.9 | 5.9 | 3828.3 | 1.485 | 26.4 |
| 80.0% | 20.0% | 61.4 | 1036.5 | 85.9 | 1.9 | 5.7 | 4029.7 | 1.476 | 29.1 |
| 85.0% | 15.0% | 65.5 | 1110.0 | 86.8 | 1.7 | 5.3 | 4267.0 | 1.463 | 32.4 |
| 90.0% | 10.0% | 70.6 | 1200.5 | 87.6 | 1.4 | 4.4 | 4553.7 | 1.447 | 36.6 |
| 95.0% | 5.0% | 77.4 | 1315.6 | 88.4 | 0.8 | 2.7 | 4913.2 | 1.426 | 42.1 |
| 100.0% | 0.0% | 86.8 | 1469.0 | 89.1 | 0.0 | 0.0 | 5375.7 | 1.396 | 49.7 |

TABLE 4M
CF₃NO₂ AND BUTANE/AIR CONDITIONING TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Butane | kPa | kPa | ° C. | Glide, ° C. | Glide, ° C. | kJ/s | COP | kg/s |
| 0.0% | 100.0% | 124.5 | 434.6 | 61.6 | 0.0 | 0.0 | 9845.2 | 4.298 | 31.5 |
| 5.0% | 95.0% | 128.1 | 447.3 | 62.1 | 0.6 | 0.8 | 10145.1 | 4.305 | 33.3 |
| 10.0% | 90.0% | 131.9 | 461.0 | 62.6 | 1.2 | 1.7 | 10468.7 | 4.312 | 35.1 |
| 15.0% | 85.0% | 136.1 | 475.9 | 63.1 | 1.8 | 2.6 | 10818.6 | 4.318 | 37.2 |
| 20.0% | 80.0% | 140.7 | 492.1 | 63.6 | 2.5 | 3.5 | 11197.8 | 4.324 | 39.5 |
| 25.0% | 75.0% | 145.7 | 509.8 | 64.2 | 3.1 | 4.4 | 11609.5 | 4.329 | 42.1 |
| 30.0% | 70.0% | 151.2 | 529.2 | 64.8 | 3.8 | 5.2 | 12058.1 | 4.333 | 44.9 |
| 35.0% | 65.0% | 157.2 | 550.5 | 65.4 | 4.4 | 6.1 | 12548.2 | 4.336 | 48.1 |
| 40.0% | 60.0% | 163.9 | 574.1 | 66.0 | 5.0 | 7.0 | 13085.1 | 4.337 | 51.7 |
| 45.0% | 55.0% | 171.4 | 600.4 | 66.6 | 5.7 | 7.8 | 13675.5 | 4.336 | 55.8 |
| 50.0% | 50.0% | 179.7 | 629.7 | 67.2 | 6.2 | 8.6 | 14327.4 | 4.333 | 60.5 |
| 55.0% | 45.0% | 189.1 | 662.8 | 67.8 | 6.8 | 9.3 | 15050.4 | 4.327 | 65.8 |
| 60.0% | 40.0% | 199.8 | 700.3 | 68.4 | 7.2 | 9.9 | 15857.0 | 4.316 | 72.1 |
| 65.0% | 35.0% | 212.1 | 743.3 | 69.0 | 7.5 | 10.4 | 16763.1 | 4.301 | 79.4 |
| 70.0% | 30.0% | 226.3 | 793.2 | 69.5 | 7.7 | 10.7 | 17790.1 | 4.280 | 88.1 |
| 75.0% | 25.0% | 243.2 | 851.9 | 70.0 | 7.7 | 10.7 | 18968.0 | 4.251 | 98.7 |
| 80.0% | 20.0% | 263.5 | 922.1 | 70.3 | 7.5 | 10.3 | 20342.0 | 4.214 | 111.8 |
| 85.0% | 15.0% | 288.8 | 1008.3 | 70.5 | 6.8 | 9.4 | 21984.7 | 4.168 | 128.4 |
| 90.0% | 10.0% | 321.8 | 1117.6 | 70.3 | 5.5 | 7.7 | 24026.7 | 4.112 | 150.7 |
| 95.0% | 5.0% | 368.1 | 1262.7 | 69.6 | 3.4 | 4.8 | 26737.2 | 4.052 | 182.6 |
| 100.0% | 0.0% | 441.0 | 1469.0 | 67.6 | 0.0 | 0.0 | 30771.3 | 4.007 | 234.3 |

TABLE 4N
CF₃NO₂ AND BUTANE/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Butane | kPa | kPa | ° C. | Glide, ° C. | Glide, ° C. | kJ/s | COP | kg/s |
| 0.0% | 100.0% | 75.7 | 434.6 | 65.8 | 0.0 | 0.0 | 5806.0 | 2.921 | 19.8 |
| 5.0% | 95.0% | 77.8 | 447.3 | 66.3 | 0.5 | 0.8 | 5981.6 | 2.926 | 20.9 |
| 10.0% | 90.0% | 80.2 | 461.0 | 66.9 | 1.1 | 1.7 | 6171.0 | 2.930 | 22.1 |
| 15.0% | 85.0% | 82.7 | 475.9 | 67.4 | 1.6 | 2.6 | 6376.1 | 2.935 | 23.4 |
| 20.0% | 80.0% | 85.4 | 492.1 | 68.0 | 2.2 | 3.5 | 6598.1 | 2.939 | 24.8 |
| 25.0% | 75.0% | 88.5 | 509.8 | 68.6 | 2.7 | 4.4 | 6839.6 | 2.942 | 26.4 |
| 30.0% | 70.0% | 91.8 | 529.2 | 69.2 | 3.3 | 5.2 | 7102.2 | 2.945 | 28.2 |
| 35.0% | 65.0% | 95.4 | 550.5 | 69.8 | 3.9 | 6.1 | 7389.5 | 2.946 | 30.2 |
| 40.0% | 60.0% | 99.5 | 574.1 | 70.5 | 4.4 | 7.0 | 7704.1 | 2.947 | 32.4 |
| 45.0% | 55.0% | 104.0 | 600.4 | 71.1 | 4.9 | 7.8 | 8050.2 | 2.946 | 34.9 |
| 50.0% | 50.0% | 109.1 | 629.7 | 71.8 | 5.4 | 8.6 | 8432.1 | 2.944 | 37.9 |
| 55.0% | 45.0% | 114.8 | 662.8 | 72.5 | 5.9 | 9.3 | 8855.8 | 2.939 | 41.2 |
| 60.0% | 40.0% | 121.2 | 700.3 | 73.1 | 6.3 | 9.9 | 9328.4 | 2.931 | 45.1 |
| 65.0% | 35.0% | 128.7 | 743.3 | 73.8 | 6.6 | 10.4 | 9859.2 | 2.920 | 49.6 |
| 70.0% | 30.0% | 137.4 | 793.2 | 74.4 | 6.8 | 10.7 | 10460.9 | 2.904 | 55.1 |
| 75.0% | 25.0% | 147.7 | 851.9 | 75.0 | 6.7 | 10.7 | 11151.6 | 2.884 | 61.7 |
| 80.0% | 20.0% | 160.1 | 922.1 | 75.5 | 6.5 | 10.3 | 11958.5 | 2.857 | 69.8 |
| 85.0% | 15.0% | 175.7 | 1008.3 | 75.8 | 5.9 | 9.4 | 12926.9 | 2.823 | 80.2 |
| 90.0% | 10.0% | 196.3 | 1117.6 | 75.8 | 4.8 | 7.7 | 14140.0 | 2.782 | 94.2 |
| 95.0% | 5.0% | 225.6 | 1262.7 | 75.2 | 2.9 | 4.8 | 15774.8 | 2.736 | 114.3 |
| 100.0% | 0.0% | 272.9 | 1469.0 | 73.3 | 0.0 | 0.0 | 18270.3 | 2.695 | 147.5 |

TABLE 4O
CF₃NO₂ AND BUTANE/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Butane | kPa | kPa | ° C. | Glide, ° C. | Glide, ° C. | kJ/s | COP | kg/s |
| 0.0% | 100.0% | 23.2 | 434.6 | 78.9 | 0.0 | 0.0 | 1681.4 | 1.553 | 6.6 |
| 5.0% | 95.0% | 23.8 | 447.3 | 79.5 | 0.4 | 0.8 | 1731.9 | 1.556 | 6.9 |
| 10.0% | 90.0% | 24.5 | 461.0 | 80.1 | 0.8 | 1.7 | 1786.4 | 1.558 | 7.3 |
| 15.0% | 85.0% | 25.3 | 475.9 | 80.7 | 1.2 | 2.6 | 1845.3 | 1.561 | 7.8 |
| 20.0% | 80.0% | 26.1 | 492.1 | 81.3 | 1.6 | 3.5 | 1909.2 | 1.563 | 8.2 |
| 25.0% | 75.0% | 27.0 | 509.8 | 81.9 | 2.0 | 4.4 | 1978.6 | 1.565 | 8.7 |
| 30.0% | 70.0% | 28.0 | 529.2 | 82.6 | 2.4 | 5.2 | 2054.2 | 1.566 | 9.3 |
| 35.0% | 65.0% | 29.2 | 550.5 | 83.3 | 2.8 | 6.1 | 2136.8 | 1.567 | 10.0 |
| 40.0% | 60.0% | 30.4 | 574.1 | 84.0 | 3.3 | 7.0 | 2227.4 | 1.567 | 10.7 |
| 45.0% | 55.0% | 31.8 | 600.4 | 84.8 | 3.6 | 7.8 | 2326.8 | 1.566 | 11.6 |
| 50.0% | 50.0% | 33.3 | 629.7 | 85.6 | 4.0 | 8.6 | 2436.7 | 1.564 | 12.5 |
| 55.0% | 45.0% | 35.1 | 662.8 | 86.4 | 4.3 | 9.3 | 2558.5 | 1.561 | 13.6 |
| 60.0% | 40.0% | 37.0 | 700.3 | 87.2 | 4.6 | 9.9 | 2694.5 | 1.556 | 14.9 |
| 65.0% | 35.0% | 39.3 | 743.3 | 88.0 | 4.8 | 10.4 | 2847.2 | 1.549 | 16.4 |
| 70.0% | 30.0% | 42.0 | 793.2 | 88.8 | 4.9 | 10.7 | 3020.6 | 1.540 | 18.2 |
| 75.0% | 25.0% | 45.2 | 851.9 | 89.6 | 4.9 | 10.7 | 3219.9 | 1.527 | 20.4 |
| 80.0% | 20.0% | 49.1 | 922.1 | 90.3 | 4.7 | 10.3 | 3453.7 | 1.511 | 23.1 |
| 85.0% | 15.0% | 54.1 | 1008.3 | 90.8 | 4.2 | 9.4 | 3736.5 | 1.489 | 26.5 |
| 90.0% | 10.0% | 60.7 | 1117.6 | 91.1 | 3.4 | 7.7 | 4096.1 | 1.463 | 31.2 |
| 95.0% | 5.0% | 70.4 | 1262.7 | 90.7 | 2.0 | 4.8 | 4593.8 | 1.431 | 38.2 |
| 100.0% | 0.0% | 86.8 | 1469.0 | 89.1 | 0.0 | 0.0 | 5375.7 | 1.396 | 49.7 |

TABLE 4P
CF₃NO₂ AND R-32/AIR CONDITIONING TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 32 | kPa | kPa | ° C. | Glide, ° C. | Glide, ° C. | kJ/s | COP | kg/s |
| 0.0% | 100.0% | 951.4 | 2794.8 | 101.5 | 0.0 | 0.0 | 61304.8 | 3.839 | 242.2 |
| 5.0% | 95.0% | 935.0 | 2754.9 | 100.2 | 0.2 | 0.2 | 60256.3 | 3.841 | 244.3 |
| 10.0% | 90.0% | 918.0 | 2713.7 | 99.0 | 0.4 | 0.4 | 59182.6 | 3.844 | 246.4 |
| 15.0% | 85.0% | 900.5 | 2671.0 | 97.6 | 0.6 | 0.6 | 58084.5 | 3.847 | 248.4 |
| 20.0% | 80.0% | 882.4 | 2626.7 | 96.3 | 0.9 | 0.8 | 56955.3 | 3.850 | 250.5 |
| 25.0% | 75.0% | 863.7 | 2580.6 | 94.9 | 1.1 | 1.0 | 55793.2 | 3.854 | 252.4 |
| 30.0% | 70.0% | 844.3 | 2532.5 | 93.5 | 1.3 | 1.2 | 54595.2 | 3.858 | 254.3 |
| 35.0% | 65.0% | 824.1 | 2482.4 | 92.1 | 1.5 | 1.4 | 53358.1 | 3.862 | 256.1 |
| 40.0% | 60.0% | 803.0 | 2429.9 | 90.6 | 1.7 | 1.6 | 52078.5 | 3.867 | 257.7 |
| 45.0% | 55.0% | 781.0 | 2374.8 | 89.1 | 1.9 | 1.7 | 50753.0 | 3.873 | 259.0 |
| 50.0% | 50.0% | 757.9 | 2316.9 | 87.6 | 2.1 | 1.9 | 49377.8 | 3.879 | 260.2 |
| 55.0% | 45.0% | 733.8 | 2255.8 | 86.0 | 2.3 | 2.1 | 47948.8 | 3.886 | 261.0 |
| 60.0% | 40.0% | 708.4 | 2191.2 | 84.4 | 2.5 | 2.2 | 46461.4 | 3.895 | 261.5 |
| 65.0% | 35.0% | 681.8 | 2122.6 | 82.8 | 2.6 | 2.4 | 44908.4 | 3.905 | 261.5 |
| 70.0% | 30.0% | 653.7 | 2049.7 | 81.0 | 2.7 | 2.5 | 43286.2 | 3.917 | 260.9 |
| 75.0% | 25.0% | 624.0 | 1971.7 | 79.2 | 2.8 | 2.6 | 41583.4 | 3.931 | 259.7 |
| 80.0% | 20.0% | 592.7 | 1887.9 | 77.3 | 2.8 | 2.5 | 39784.9 | 3.948 | 257.7 |
| 85.0% | 15.0% | 559.2 | 1797.4 | 75.2 | 2.6 | 2.4 | 37866.0 | 3.967 | 254.6 |
| 90.0% | 10.0% | 523.4 | 1698.9 | 73.0 | 2.2 | 2.0 | 35785.3 | 3.987 | 250.1 |
| 95.0% | 5.0% | 484.4 | 1590.4 | 70.5 | 1.4 | 1.3 | 33464.9 | 4.003 | 243.6 |
| 100.0% | 0.0% | 441.0 | 1469.0 | 67.6 | 0.0 | 0.0 | 30771.3 | 4.007 | 234.3 |

TABLE 4Q
CF₃NO₂ AND R-32/MEDIUM TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 32 | kPa | kPa | ° C. | Glide, ° C. | Glide, ° C. | kJ/s | COP | kg/s |
| 0.0% | 100.0% | 624.1 | 2794.8 | 122.4 | 0.0 | 0.0 | 39866.0 | 2.625 | 159.8 |
| 5.0% | 95.0% | 612.4 | 2754.9 | 120.4 | 0.2 | 0.2 | 39089.5 | 2.626 | 161.0 |
| 10.0% | 90.0% | 600.4 | 2713.7 | 118.4 | 0.4 | 0.4 | 38295.0 | 2.628 | 162.1 |
| 15.0% | 85.0% | 588.0 | 2671.0 | 116.4 | 0.6 | 0.6 | 37483.7 | 2.629 | 163.3 |
| 20.0% | 80.0% | 575.2 | 2626.7 | 114.3 | 0.9 | 0.8 | 36650.6 | 2.630 | 164.3 |
| 25.0% | 75.0% | 561.9 | 2580.6 | 112.2 | 1.1 | 1.0 | 35794.7 | 2.632 | 165.4 |
| 30.0% | 70.0% | 548.2 | 2532.5 | 110.1 | 1.3 | 1.2 | 34914.0 | 2.633 | 166.3 |
| 35.0% | 65.0% | 534.0 | 2482.4 | 107.9 | 1.5 | 1.4 | 34006.5 | 2.635 | 167.1 |
| 40.0% | 60.0% | 519.1 | 2429.9 | 105.8 | 1.7 | 1.6 | 33070.4 | 2.636 | 167.8 |
| 45.0% | 55.0% | 503.7 | 2374.8 | 103.5 | 1.9 | 1.7 | 32103.8 | 2.638 | 168.4 |
| 50.0% | 50.0% | 487.5 | 2316.9 | 101.3 | 2.1 | 1.9 | 31103.8 | 2.640 | 168.8 |
| 55.0% | 45.0% | 470.7 | 2255.8 | 99.0 | 2.3 | 2.1 | 30070.5 | 2.643 | 168.9 |
| 60.0% | 40.0% | 453.1 | 2191.2 | 96.6 | 2.5 | 2.2 | 29001.3 | 2.646 | 168.8 |
| 65.0% | 35.0% | 434.6 | 2122.6 | 94.2 | 2.6 | 2.4 | 27894.2 | 2.650 | 168.3 |
| 70.0% | 30.0% | 415.4 | 2049.7 | 91.6 | 2.8 | 2.5 | 26746.4 | 2.655 | 167.5 |
| 75.0% | 25.0% | 395.1 | 1971.7 | 89.0 | 2.8 | 2.6 | 25553.4 | 2.662 | 166.2 |
| 80.0% | 20.0% | 373.9 | 1887.9 | 86.3 | 2.8 | 2.5 | 24307.4 | 2.670 | 164.5 |
| 85.0% | 15.0% | 351.4 | 1797.4 | 83.4 | 2.6 | 2.4 | 22994.5 | 2.680 | 162.0 |
| 90.0% | 10.0% | 327.5 | 1698.9 | 80.3 | 2.2 | 2.0 | 21588.8 | 2.690 | 158.7 |
| 95.0% | 5.0% | 301.6 | 1590.4 | 77.0 | 1.4 | 1.3 | 20043.6 | 2.697 | 154.0 |
| 100.0% | 0.0% | 272.9 | 1469.0 | 73.3 | 0.0 | 0.0 | 18270.3 | 2.695 | 147.5 |

TABLE 4R
CF₃NO₂ AND R-32/LOW TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 32 | kPa | kPa | ° C. | Glide, ° C. | Glide, ° C. | kJ/s | COP | kg/s |
| 0.0% | 100.0% | 231.1 | 2794.8 | 175.4 | 0.0 | 0.0 | 14725.4 | 1.418 | 61.7 |
| 5.0% | 95.0% | 225.9 | 2754.9 | 171.8 | 0.2 | 0.2 | 14351.9 | 1.417 | 61.9 |
| 10.0% | 90.0% | 220.5 | 2713.7 | 168.0 | 0.4 | 0.4 | 13971.6 | 1.417 | 62.1 |
| 15.0% | 85.0% | 214.9 | 2671.0 | 164.3 | 0.6 | 0.6 | 13582.9 | 1.417 | 62.2 |
| 20.0% | 80.0% | 209.2 | 2626.7 | 160.5 | 0.8 | 0.8 | 13185.9 | 1.416 | 62.4 |
| 25.0% | 75.0% | 203.4 | 2580.6 | 156.6 | 1.1 | 1.0 | 12779.5 | 1.416 | 62.5 |
| 30.0% | 70.0% | 197.3 | 2532.5 | 152.7 | 1.3 | 1.2 | 12363.4 | 1.415 | 62.5 |
| 35.0% | 65.0% | 191.0 | 2482.4 | 148.8 | 1.5 | 1.4 | 11937.2 | 1.413 | 62.5 |
| 40.0% | 60.0% | 184.5 | 2429.9 | 144.8 | 1.7 | 1.6 | 11500.2 | 1.412 | 62.4 |
| 45.0% | 55.0% | 177.8 | 2374.8 | 140.8 | 1.9 | 1.7 | 11053.2 | 1.411 | 62.2 |
| 50.0% | 50.0% | 170.9 | 2316.9 | 136.7 | 2.1 | 1.9 | 10596.3 | 1.409 | 61.9 |
| 55.0% | 45.0% | 163.7 | 2255.8 | 132.5 | 2.3 | 2.1 | 10130.0 | 1.407 | 61.5 |
| 60.0% | 40.0% | 156.3 | 2191.2 | 128.3 | 2.5 | 2.2 | 9654.9 | 1.406 | 61.0 |
| 65.0% | 35.0% | 148.7 | 2122.6 | 124.0 | 2.7 | 2.4 | 9171.9 | 1.404 | 60.4 |
| 70.0% | 30.0% | 140.9 | 2049.7 | 119.5 | 2.8 | 2.5 | 8681.3 | 1.404 | 59.6 |
| 75.0% | 25.0% | 132.8 | 1971.7 | 115.0 | 2.9 | 2.6 | 8182.9 | 1.403 | 58.7 |
| 80.0% | 20.0% | 124.5 | 1887.9 | 110.2 | 2.8 | 2.5 | 7675.2 | 1.404 | 57.7 |
| 85.0% | 15.0% | 115.9 | 1797.4 | 105.3 | 2.6 | 2.4 | 7154.0 | 1.405 | 56.3 |
| 90.0% | 10.0% | 106.9 | 1698.9 | 100.2 | 2.2 | 2.0 | 6610.8 | 1.405 | 54.7 |
| 95.0% | 5.0% | 97.4 | 1590.4 | 94.8 | 1.5 | 1.3 | 6029.0 | 1.404 | 52.6 |
| 100.0% | 0.0% | 86.8 | 1469.0 | 89.1 | 0.0 | 0.0 | 5375.7 | 1.396 | 49.7 |

TABLE 4S
CF₃NO₂ AND R-152a/AIR CONDITIONING TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 152a | kPa | kPa | ° C. | Glide, ° C. | Glide, ° C. | kJ/s | COP | kg/s |
| 0.0% | 100.0% | 314.8 | 1036.8 | 79.8 | 0.0 | 0.0 | 23598.6 | 4.198 | 94.3 |
| 5.0% | 95.0% | 318.2 | 1047.8 | 79.4 | 0.1 | 0.1 | 23799.2 | 4.194 | 97.4 |
| 10.0% | 90.0% | 321.7 | 1059.3 | 78.9 | 0.2 | 0.2 | 24009.3 | 4.190 | 100.7 |
| 15.0% | 85.0% | 325.4 | 1071.5 | 78.4 | 0.2 | 0.3 | 24229.5 | 4.186 | 104.3 |
| 20.0% | 80.0% | 329.4 | 1084.3 | 77.9 | 0.3 | 0.4 | 24460.7 | 4.181 | 108.0 |
| 25.0% | 75.0% | 333.5 | 1097.9 | 77.4 | 0.4 | 0.5 | 24703.8 | 4.176 | 112.1 |
| 30.0% | 70.0% | 337.9 | 1112.4 | 76.9 | 0.4 | 0.6 | 24959.6 | 4.170 | 116.4 |
| 35.0% | 65.0% | 342.5 | 1127.7 | 76.4 | 0.5 | 0.7 | 25229.3 | 4.164 | 121.0 |
| 40.0% | 60.0% | 347.5 | 1144.0 | 75.8 | 0.5 | 0.7 | 25514.2 | 4.157 | 126.0 |
| 45.0% | 55.0% | 352.7 | 1161.4 | 75.2 | 0.6 | 0.8 | 25815.5 | 4.150 | 131.4 |
| 50.0% | 50.0% | 358.3 | 1180.0 | 74.6 | 0.6 | 0.8 | 26134.8 | 4.142 | 137.2 |
| 55.0% | 45.0% | 364.3 | 1199.9 | 74.0 | 0.6 | 0.9 | 26473.7 | 4.133 | 143.5 |
| 60.0% | 40.0% | 370.6 | 1221.3 | 73.4 | 0.6 | 0.9 | 26834.3 | 4.124 | 150.3 |
| 65.0% | 35.0% | 377.5 | 1244.3 | 72.7 | 0.6 | 0.9 | 27218.5 | 4.113 | 157.8 |
| 70.0% | 30.0% | 384.8 | 1269.1 | 72.1 | 0.5 | 0.8 | 27628.7 | 4.102 | 165.9 |
| 75.0% | 25.0% | 392.6 | 1295.9 | 71.4 | 0.5 | 0.8 | 28067.4 | 4.090 | 174.8 |
| 80.0% | 20.0% | 401.1 | 1324.9 | 70.6 | 0.4 | 0.7 | 28537.3 | 4.076 | 184.6 |
| 85.0% | 15.0% | 410.1 | 1356.5 | 69.9 | 0.3 | 0.6 | 29040.8 | 4.061 | 195.3 |
| 90.0% | 10.0% | 419.8 | 1390.8 | 69.1 | 0.2 | 0.4 | 29580.3 | 4.045 | 207.1 |
| 95.0% | 5.0% | 430.1 | 1428.2 | 68.4 | 0.1 | 0.2 | 30157.2 | 4.027 | 220.1 |
| 100.0% | 0.0% | 441.0 | 1469.0 | 67.6 | 0.0 | 0.0 | 30771.3 | 4.007 | 234.3 |

TABLE 4T
CF₃NO₂ AND R-152a/MEDIUM TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 152a | kPa | kPa | ° C. | Glide, ° C. | Glide, ° C. | kJ/s | COP | kg/s |
| 0.0% | 100.0% | 196.1 | 1036.8 | 90.8 | 0.0 | 0.0 | 14468.3 | 2.872 | 60.1 |
| 5.0% | 95.0% | 198.3 | 1047.8 | 90.1 | 0.1 | 0.1 | 14581.6 | 2.869 | 62.1 |
| 10.0% | 90.0% | 200.5 | 1059.3 | 89.4 | 0.1 | 0.2 | 14699.9 | 2.865 | 64.2 |
| 15.0% | 85.0% | 202.8 | 1071.5 | 88.7 | 0.2 | 0.3 | 14822.8 | 2.861 | 66.4 |
| 20.0% | 80.0% | 205.2 | 1084.3 | 88.0 | 0.3 | 0.4 | 14952.0 | 2.856 | 68.8 |
| 25.0% | 75.0% | 207.8 | 1097.9 | 87.3 | 0.3 | 0.5 | 15087.4 | 2.851 | 71.4 |
| 30.0% | 70.0% | 210.6 | 1112.4 | 86.5 | 0.4 | 0.6 | 15229.4 | 2.846 | 74.1 |
| 35.0% | 65.0% | 213.4 | 1127.7 | 85.7 | 0.4 | 0.7 | 15378.6 | 2.840 | 77.1 |
| 40.0% | 60.0% | 216.5 | 1144.0 | 84.9 | 0.4 | 0.7 | 15535.5 | 2.834 | 80.2 |
| 45.0% | 55.0% | 219.8 | 1161.4 | 84.1 | 0.5 | 0.8 | 15700.8 | 2.827 | 83.6 |
| 50.0% | 50.0% | 223.2 | 1180.0 | 83.2 | 0.5 | 0.8 | 15875.7 | 2.820 | 87.3 |
| 55.0% | 45.0% | 226.9 | 1199.9 | 82.3 | 0.5 | 0.9 | 16059.7 | 2.811 | 91.2 |
| 60.0% | 40.0% | 230.8 | 1221.3 | 81.4 | 0.5 | 0.9 | 16254.3 | 2.803 | 95.5 |
| 65.0% | 35.0% | 235.0 | 1244.3 | 80.4 | 0.5 | 0.9 | 16460.4 | 2.793 | 100.2 |
| 70.0% | 30.0% | 239.5 | 1269.1 | 79.5 | 0.4 | 0.8 | 16678.8 | 2.783 | 105.3 |
| 75.0% | 25.0% | 244.3 | 1295.9 | 78.5 | 0.4 | 0.8 | 16910.4 | 2.771 | 110.9 |
| 80.0% | 20.0% | 249.4 | 1324.9 | 77.5 | 0.3 | 0.7 | 17155.8 | 2.759 | 116.9 |
| 85.0% | 15.0% | 254.8 | 1356.5 | 76.4 | 0.3 | 0.6 | 17415.4 | 2.745 | 123.6 |
| 90.0% | 10.0% | 260.6 | 1390.8 | 75.4 | 0.2 | 0.4 | 17689.0 | 2.730 | 130.9 |
| 95.0% | 5.0% | 266.7 | 1428.2 | 74.3 | 0.1 | 0.2 | 17975.0 | 2.714 | 138.9 |
| 100.0% | 0.0% | 272.9 | 1469.0 | 73.3 | 0.0 | 0.0 | 18270.3 | 2.695 | 147.5 |

TABLE 4U
CF₃NO₂ AND R-152a/LOW TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 152a | kPa | kPa | ° C. | Glide, ° C. | Glide, ° C. | kJ/s | COP | kg/s |
| 0.0% | 100.0% | 63.8 | 1036.8 | 119.5 | 0.0 | 0.0 | 4608.6 | 1.551 | 20.9 |
| 5.0% | 95.0% | 64.5 | 1047.8 | 118.3 | 0.1 | 0.1 | 4637.6 | 1.548 | 21.6 |
| 10.0% | 90.0% | 65.2 | 1059.3 | 117.1 | 0.1 | 0.2 | 4667.6 | 1.544 | 22.3 |
| 15.0% | 85.0% | 65.9 | 1071.5 | 115.8 | 0.1 | 0.3 | 4698.7 | 1.541 | 23.1 |
| 20.0% | 80.0% | 66.7 | 1084.3 | 114.6 | 0.2 | 0.4 | 4730.8 | 1.537 | 23.9 |
| 25.0% | 75.0% | 67.6 | 1097.9 | 113.2 | 0.2 | 0.5 | 4764.1 | 1.532 | 24.8 |
| 30.0% | 70.0% | 68.4 | 1112.4 | 111.9 | 0.3 | 0.6 | 4798.6 | 1.528 | 25.7 |
| 35.0% | 65.0% | 69.4 | 1127.7 | 110.5 | 0.3 | 0.7 | 4834.3 | 1.522 | 26.7 |
| 40.0% | 60.0% | 70.3 | 1144.0 | 109.0 | 0.3 | 0.7 | 4871.3 | 1.517 | 27.8 |
| 45.0% | 55.0% | 71.4 | 1161.4 | 107.6 | 0.3 | 0.8 | 4909.7 | 1.511 | 29.0 |
| 50.0% | 50.0% | 72.5 | 1180.0 | 106.0 | 0.3 | 0.8 | 4949.4 | 1.504 | 30.2 |
| 55.0% | 45.0% | 73.6 | 1199.9 | 104.5 | 0.3 | 0.9 | 4990.5 | 1.497 | 31.5 |
| 60.0% | 40.0% | 74.8 | 1221.3 | 102.9 | 0.3 | 0.9 | 5033.0 | 1.490 | 33.0 |
| 65.0% | 35.0% | 76.1 | 1244.3 | 101.3 | 0.3 | 0.9 | 5076.9 | 1.481 | 34.6 |
| 70.0% | 30.0% | 77.5 | 1269.1 | 99.6 | 0.3 | 0.8 | 5121.8 | 1.472 | 36.3 |
| 75.0% | 25.0% | 78.9 | 1295.9 | 97.9 | 0.2 | 0.8 | 5167.6 | 1.462 | 38.1 |
| 80.0% | 20.0% | 80.4 | 1324.9 | 96.1 | 0.2 | 0.7 | 5213.7 | 1.452 | 40.1 |
| 85.0% | 15.0% | 82.0 | 1356.5 | 94.4 | 0.1 | 0.6 | 5259.3 | 1.440 | 42.3 |
| 90.0% | 10.0% | 83.6 | 1390.8 | 92.6 | 0.1 | 0.4 | 5303.0 | 1.426 | 44.6 |
| 95.0% | 5.0% | 85.2 | 1428.2 | 90.8 | 0.0 | 0.2 | 5342.8 | 1.412 | 47.1 |
| 100.0% | 0.0% | 86.8 | 1469.0 | 89.1 | 0.0 | 0.0 | 5375.7 | 1.396 | 49.7 |

TABLE 4V
CF₃NO₂ AND R-134a/AIR CONDITIONING TEMPERATURE CONDITIONS

| | | | | | | | | | Mass |
|---|---|---|---|---|---|---|---|---|---|
| Wt % | wt % r- | P_evap, | P_cond, | T_comp_exit, | Evap | Cond | Capacity, | | Flow, |
| CF3NO2 | 134a | kPa | kPa | ° C. | Glide, ° C. | Glide, ° C. | kJ/s | COP | kg/s |
| 0.0% | 100.0% | 349.7 | 1159.9 | 68.9 | 0.0 | 0.0 | 25113.6 | 4.103 | 162.8 |
| 5.0% | 95.0% | 355.0 | 1176.2 | 68.8 | 0.1 | 0.1 | 25427.6 | 4.098 | 166.4 |
| 10.0% | 90.0% | 360.4 | 1192.6 | 68.8 | 0.2 | 0.2 | 25739.7 | 4.093 | 170.0 |
| 15.0% | 85.0% | 365.7 | 1209.0 | 68.7 | 0.2 | 0.3 | 26050.1 | 4.088 | 173.6 |
| 20.0% | 80.0% | 371.1 | 1225.4 | 68.6 | 0.3 | 0.4 | 26359.0 | 4.082 | 177.4 |
| 25.0% | 75.0% | 376.5 | 1241.8 | 68.5 | 0.3 | 0.4 | 26666.4 | 4.077 | 181.1 |
| 30.0% | 70.0% | 381.8 | 1258.3 | 68.4 | 0.3 | 0.4 | 26972.4 | 4.071 | 184.9 |
| 35.0% | 65.0% | 387.1 | 1274.7 | 68.3 | 0.3 | 0.4 | 27276.8 | 4.066 | 188.8 |
| 40.0% | 60.0% | 392.4 | 1291.1 | 68.2 | 0.3 | 0.5 | 27579.4 | 4.060 | 192.6 |
| 45.0% | 55.0% | 397.7 | 1307.5 | 68.2 | 0.3 | 0.4 | 27879.9 | 4.055 | 196.5 |
| 50.0% | 50.0% | 402.8 | 1323.8 | 68.1 | 0.3 | 0.4 | 28177.8 | 4.050 | 200.4 |
| 55.0% | 45.0% | 407.9 | 1339.9 | 68.0 | 0.3 | 0.4 | 28472.6 | 4.044 | 204.3 |
| 60.0% | 40.0% | 412.8 | 1355.9 | 67.9 | 0.2 | 0.4 | 28763.5 | 4.039 | 208.2 |
| 65.0% | 35.0% | 417.5 | 1371.7 | 67.8 | 0.2 | 0.3 | 29049.4 | 4.034 | 212.0 |
| 70.0% | 30.0% | 422.1 | 1387.2 | 67.7 | 0.2 | 0.3 | 29329.1 | 4.030 | 215.8 |
| 75.0% | 25.0% | 426.3 | 1402.4 | 67.6 | 0.1 | 0.2 | 29601.0 | 4.025 | 219.4 |
| 80.0% | 20.0% | 430.3 | 1417.1 | 67.6 | 0.1 | 0.2 | 29863.4 | 4.021 | 222.9 |
| 85.0% | 15.0% | 433.8 | 1431.3 | 67.6 | 0.0 | 0.1 | 30114.1 | 4.017 | 226.2 |
| 90.0% | 10.0% | 436.8 | 1444.8 | 67.6 | 0.0 | 0.1 | 30350.8 | 4.013 | 229.3 |
| 95.0% | 5.0% | 439.2 | 1457.4 | 67.6 | 0.0 | 0.0 | 30570.8 | 4.010 | 232.0 |
| 100.0% | 0.0% | 441.0 | 1469.0 | 67.6 | 0.0 | 0.0 | 30771.3 | 4.007 | 234.3 |

TABLE 4W
CF₃NO₂ AND R-134A/MEDIUM TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 134a | kPa | kPa | ° C. | Glide, ° C. | Glide, ° C. | kJ/s | COP | kg/s |
| 0.0% | 100.0% | 216.9 | 1159.9 | 75.3 | 0.0 | 0.0 | 15062.1 | 2.776 | 103.1 |
| 5.0% | 95.0% | 220.4 | 1176.2 | 75.2 | 0.1 | 0.1 | 15251.3 | 2.772 | 105.4 |
| 10.0% | 90.0% | 223.8 | 1192.6 | 75.0 | 0.2 | 0.2 | 15438.4 | 2.768 | 107.7 |
| 15.0% | 85.0% | 227.3 | 1209.0 | 74.9 | 0.2 | 0.3 | 15624.6 | 2.763 | 110.1 |
| 20.0% | 80.0% | 230.7 | 1225.4 | 74.8 | 0.3 | 0.4 | 15809.7 | 2.759 | 112.4 |
| 25.0% | 75.0% | 234.1 | 1241.8 | 74.6 | 0.3 | 0.4 | 15993.5 | 2.754 | 114.8 |
| 30.0% | 70.0% | 237.6 | 1258.3 | 74.5 | 0.3 | 0.4 | 16175.9 | 2.750 | 117.3 |
| 35.0% | 65.0% | 241.0 | 1274.7 | 74.3 | 0.3 | 0.4 | 16356.9 | 2.745 | 119.7 |
| 40.0% | 60.0% | 244.3 | 1291.1 | 74.2 | 0.3 | 0.5 | 16536.1 | 2.741 | 122.2 |
| 45.0% | 55.0% | 247.7 | 1307.5 | 74.0 | 0.3 | 0.4 | 16713.3 | 2.736 | 124.7 |
| 50.0% | 50.0% | 250.9 | 1323.8 | 73.9 | 0.3 | 0.4 | 16888.7 | 2.732 | 127.2 |
| 55.0% | 45.0% | 254.1 | 1339.9 | 73.8 | 0.2 | 0.4 | 17060.2 | 2.727 | 129.6 |
| 60.0% | 40.0% | 257.2 | 1355.9 | 73.6 | 0.2 | 0.4 | 17227.9 | 2.723 | 132.1 |
| 65.0% | 35.0% | 260.1 | 1371.7 | 73.5 | 0.2 | 0.3 | 17390.6 | 2.719 | 134.5 |
| 70.0% | 30.0% | 262.9 | 1387.2 | 73.4 | 0.1 | 0.3 | 17547.4 | 2.715 | 136.8 |
| 75.0% | 25.0% | 265.4 | 1402.4 | 73.3 | 0.1 | 0.2 | 17696.7 | 2.711 | 139.0 |
| 80.0% | 20.0% | 267.7 | 1417.1 | 73.2 | 0.1 | 0.2 | 17837.0 | 2.707 | 141.1 |
| 85.0% | 15.0% | 269.7 | 1431.3 | 73.2 | 0.0 | 0.1 | 17966.4 | 2.704 | 143.1 |
| 90.0% | 10.0% | 271.2 | 1444.8 | 73.2 | 0.0 | 0.1 | 18083.1 | 2.701 | 144.8 |
| 95.0% | 5.0% | 272.4 | 1457.4 | 73.2 | 0.0 | 0.0 | 18185.0 | 2.698 | 146.3 |
| 100.0% | 0.0% | 272.9 | 1469.0 | 73.3 | 0.0 | 0.0 | 18270.3 | 2.695 | 147.5 |

TABLE 4X
CF₃NO₂ AND R-134a/LOW TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 134a | kPa | kPa | ° C. | Glide, ° C. | Glide, ° C. | kJ/s | COP | kg/s |
| 0.0% | 100.0% | 69.5 | 1159.9 | 93.0 | 0.0 | 0.0 | 4547.8 | 1.460 | 35.2 |
| 5.0% | 95.0% | 70.7 | 1176.2 | 92.7 | 0.1 | 0.1 | 4605.8 | 1.457 | 36.0 |
| 10.0% | 90.0% | 71.9 | 1192.6 | 92.5 | 0.1 | 0.2 | 4663.1 | 1.454 | 36.9 |
| 15.0% | 85.0% | 73.1 | 1209.0 | 92.2 | 0.2 | 0.3 | 4719.9 | 1.450 | 37.7 |
| 20.0% | 80.0% | 74.3 | 1225.4 | 91.9 | 0.2 | 0.4 | 4776.0 | 1.447 | 38.5 |
| 25.0% | 75.0% | 75.5 | 1241.8 | 91.6 | 0.2 | 0.4 | 4831.4 | 1.443 | 39.4 |
| 30.0% | 70.0% | 76.7 | 1258.3 | 91.3 | 0.2 | 0.4 | 4886.0 | 1.440 | 40.3 |
| 35.0% | 65.0% | 77.8 | 1274.7 | 91.0 | 0.2 | 0.4 | 4939.8 | 1.436 | 41.1 |
| 40.0% | 60.0% | 79.0 | 1291.1 | 90.7 | 0.2 | 0.5 | 4992.5 | 1.432 | 42.0 |
| 45.0% | 55.0% | 80.1 | 1307.5 | 90.4 | 0.2 | 0.4 | 5044.0 | 1.429 | 42.9 |
| 50.0% | 50.0% | 81.2 | 1323.8 | 90.2 | 0.2 | 0.4 | 5093.9 | 1.426 | 43.7 |
| 55.0% | 45.0% | 82.3 | 1339.9 | 89.9 | 0.2 | 0.4 | 5141.8 | 1.422 | 44.6 |
| 60.0% | 40.0% | 83.3 | 1355.9 | 89.6 | 0.1 | 0.4 | 5187.3 | 1.419 | 45.4 |
| 65.0% | 35.0% | 84.2 | 1371.7 | 89.4 | 0.1 | 0.3 | 5229.6 | 1.415 | 46.2 |
| 70.0% | 30.0% | 85.0 | 1387.2 | 89.2 | 0.1 | 0.3 | 5268.3 | 1.412 | 47.0 |
| 75.0% | 25.0% | 85.7 | 1402.4 | 89.1 | 0.0 | 0.2 | 5302.3 | 1.409 | 47.7 |
| 80.0% | 20.0% | 86.3 | 1417.1 | 88.9 | 0.0 | 0.2 | 5331.1 | 1.406 | 48.3 |
| 85.0% | 15.0% | 86.7 | 1431.3 | 88.9 | 0.0 | 0.1 | 5353.4 | 1.403 | 48.8 |
| 90.0% | 10.0% | 87.0 | 1444.8 | 88.9 | 0.0 | 0.1 | 5368.9 | 1.400 | 49.3 |
| 95.0% | 5.0% | 87.0 | 1457.4 | 88.9 | 0.0 | 0.0 | 5376.3 | 1.398 | 49.6 |
| 100.0% | 0.0% | 86.8 | 1469.0 | 89.1 | 0.0 | 0.0 | 5375.7 | 1.396 | 49.7 |

TABLE 4Y
CF₃NO₂ AND R-125/AIR CONDITIONING TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 125 | kPa | kPa | ° C. | Glide, ° C. | Glide, ° C. | kJ/s | COP | kg/s |
| 0.0% | 100.0% | 782.9 | 2260.7 | 62.4 | 0.0 | 0.0 | 41635.3 | 3.593 | 461.8 |
| 5.0% | 95.0% | 762.3 | 2216.3 | 62.7 | 0.2 | 0.2 | 41168.4 | 3.618 | 446. 8 |
| 10.0% | 90.0% | 742.3 | 2172.6 | 63.0 | 0.4 | 0.3 | 40692.4 | 3.643 | 432.3 |
| 15.0% | 85.0% | 722.9 | 2129.6 | 63.2 | 0.5 | 0.5 | 40211.5 | 3.667 | 418.5 |
| 20.0% | 80.0% | 704.0 | 2087.4 | 63.5 | 0.7 | 0.6 | 39722.6 | 3.690 | 405.1 |
| 25.0% | 75.0% | 685.7 | 2045.9 | 63.8 | 0.8 | 0.7 | 39227.3 | 3.713 | 392.3 |
| 30.0% | 70.0% | 667.8 | 2005.1 | 64.1 | 1.0 | 0.8 | 38725.9 | 3.735 | 379.9 |
| 35.0% | 65.0% | 650.3 | 1964.8 | 64.4 | 1.1 | 0.9 | 38218.4 | 3.757 | 367.9 |
| 40.0% | 60.0% | 633.1 | 1925.2 | 64.7 | 1.2 | 1.0 | 37704.9 | 3.778 | 356. 2 |
| 45.0% | 55.0% | 616.4 | 1886.1 | 64.9 | 1.3 | 1.1 | 37185.4 | 3.799 | 345.0 |
| 50.0% | 50.0% | 599.9 | 1847.5 | 65.2 | 1.4 | 1.1 | 36659.7 | 3.820 | 334.0 |
| 55.0% | 45.0% | 583.7 | 1809.3 | 65.5 | 1.4 | 1.1 | 36127.7 | 3.840 | 323. 3 |
| 60.0% | 40.0% | 567.7 | 1771.5 | 65.8 | 1.4 | 1.2 | 35588.9 | 3.860 | 312. 9 |
| 65.0% | 35.0% | 551.9 | 1734.0 | 66.0 | 1.5 | 1.1 | 35042.9 | 3.880 | 302.7 |
| 70.0% | 30.0% | 536.3 | 1696.7 | 66.3 | 1.4 | 1.1 | 34488.7 | 3.899 | 292.7 |
| 75.0% | 25.0% | 520.8 | 1659.4 | 66.6 | 1.4 | 1.0 | 33924.9 | 3.919 | 282. 9 |
| 80.0% | 20.0% | 505.2 | 1622.2 | 66.8 | 1.3 | 0.9 | 33348.9 | 3.938 | 273.2 |
| 85.0% | 15.0% | 489.7 | 1584.8 | 67.0 | 1.1 | 0.8 | 32756.2 | 3.957 | 263.6 |
| 90.0% | 10.0% | 473.9 | 1547.0 | 67.3 | 0.9 | 0.6 | 32139.3 | 3.976 | 254. 0 |
| 95.0% | 5.0% | 457.8 | 1508.6 | 67.5 | 0.5 | 0.3 | 31483.9 | 3.993 | 244. 2 |
| 100.0% | 0.0% | 441.0 | 1469.0 | 67.6 | 0.0 | 0.0 | 30771.3 | 4.007 | 234.3 |

TABLE 4Z
CF₃NO₂ AND R-125/MEDIUM TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 152 | kPa | kPa | ° C. | Glide, ° C. | Glide, ° C. | kJ/s | COP | kg/s |
| 0.0% | 100.0% | 516.5 | 2260.7 | 65.2 | 0.0 | 0.0 | 25457.4 | 2.366 | 305.5 |
| 5.0% | 95.0% | 501.2 | 2216.3 | 65.7 | 0.2 | 0.2 | 25142.7 | 2.386 | 294.8 |
| 10.0% | 90.0% | 486.5 | 2172.6 | 66.1 | 0.4 | 0.3 | 24822.7 | 2.404 | 284.6 |
| 15.0% | 85.0% | 472.3 | 2129.6 | 66.5 | 0.5 | 0.5 | 24501.1 | 2.423 | 274.9 |
| 20.0% | 80.0% | 458.6 | 2087.4 | 66.9 | 0.7 | 0.6 | 24174.9 | 2.441 | 265.5 |
| 25.0% | 75.0% | 445.2 | 2045.9 | 67.3 | 0.8 | 0.7 | 23845.2 | 2.459 | 256.4 |
| 30.0% | 70.0% | 432.3 | 2005.1 | 67.7 | 1.0 | 0.8 | 23511.9 | 2.476 | 247.8 |
| 35.0% | 65.0% | 419.7 | 1964.8 | 68.1 | 1.1 | 0.9 | 23175.2 | 2.493 | 239.4 |
| 40.0% | 60.0% | 407.4 | 1925.2 | 68.6 | 1.2 | 1.0 | 22834.8 | 2.510 | 231.3 |
| 45.0% | 55.0% | 395.4 | 1886.1 | 69.0 | 1.3 | 1.1 | 22490.8 | 2.526 | 223.4 |
| 50.0% | 50.0% | 383.7 | 1847.5 | 69.4 | 1.4 | 1.1 | 22143.0 | 2.542 | 215.8 |
| 55.0% | 45.0% | 372.2 | 1809.3 | 69.8 | 1.5 | 1.1 | 21791.5 | 2.558 | 208.4 |
| 60.0% | 40.0% | 360.9 | 1771.5 | 70.2 | 1.5 | 1.2 | 21436.1 | 2.574 | 201.2 |
| 65.0% | 35.0% | 349.8 | 1734.0 | 70.6 | 1.5 | 1.1 | 21076.6 | 2.589 | 194.2 |
| 70.0% | 30.0% | 338.9 | 1696.7 | 71.0 | 1.5 | 1.1 | 20712.5 | 2.605 | 187.3 |
| 75.0% | 25.0% | 328.0 | 1659.4 | 71.5 | 1.5 | 1.0 | 20343.1 | 2.621 | 180.6 |
| 80.0% | 20.0% | 317.3 | 1622.2 | 71.9 | 1.4 | 0.9 | 19966.5 | 2.637 | 174.0 |
| 85.0% | 15.0% | 306.5 | 1584.8 | 72.2 | 1.2 | 0.8 | 19579.4 | 2.652 | 167.4 |
| 90.0% | 10.0% | 295.7 | 1547.0 | 72.6 | 0.9 | 0.6 | 19175.9 | 2.668 | 160.9 |
| 95.0% | 5.0% | 284.5 | 1508.6 | 73.0 | 0.6 | 0.3 | 18745.4 | 2.683 | 154.3 |
| 100.0% | 0.0% | 272.9 | 1469.0 | 73.3 | 0.0 | 0.0 | 18270.3 | 2.695 | 147.5 |

TABLE 4AA
CF₃NO₂ AND R-125/LOW TEMPERATURE CONDITIONS

| | | | | | | | | | Mass |
|---|---|---|---|---|---|---|---|---|---|
| Wt % | wt % R- | P_evap, | P_cond, | T_comp_exit, | Evap | Cond | Capacity, | | Flow, |
| CF3NO2 | 152 | kPa | kPa | ° C. | Glide, ° C. | Glide, ° C. | kJ/s | COP | kg/s |
| 0.0% | 100.0% | 193.0 | 2260.7 | 73.5 | 0.0 | 0.0 | 8125.8 | 1.157 | 118.6 |
| 5.0% | 95.0% | 185.6 | 2216.3 | 74.3 | 0.2 | 0.2 | 7995.6 | 1.171 | 113.6 |
| 10.0% | 90.0% | 178.6 | 2172.6 | 75.1 | 0.3 | 0.3 | 7864.8 | 1.184 | 108.8 |
| 15.0% | 85.0% | 171.9 | 2129.6 | 75.9 | 0.5 | 0.5 | 7733.6 | 1.197 | 104.3 |
| 20.0% | 80.0% | 165.5 | 2087.4 | 76.6 | 0.6 | 0.6 | 7601.8 | 1.209 | 100.0 |
| 25.0% | 75.0% | 159.3 | 2045.9 | 77.4 | 0.8 | 0.7 | 7469.3 | 1.222 | 95.9 |
| 30.0% | 70.0% | 153.4 | 2005.1 | 78.2 | 0.9 | 0.8 | 7336.1 | 1.234 | 92.0 |
| 35.0% | 65.0% | 147.7 | 1964.8 | 79.0 | 1.1 | 0.9 | 7202.3 | 1.246 | 88.2 |
| 40.0% | 60.0% | 142.2 | 1925.2 | 79.8 | 1.2 | 1.0 | 7067.4 | 1.257 | 84.6 |
| 45.0% | 55.0% | 137.0 | 1886.1 | 80.6 | 1.3 | 1.1 | 6932.3 | 1.269 | 81.2 |
| 50.0% | 50.0% | 131.8 | 1847.5 | 81.4 | 1.4 | 1.1 | 6797.0 | 1.280 | 77.9 |
| 55.0% | 45.0% | 126.9 | 1809.3 | 82.2 | 1.5 | 1.1 | 6661.6 | 1.292 | 74.7 |
| 60.0% | 40.0% | 122.1 | 1771.5 | 83.0 | 1.6 | 1.2 | 6526.4 | 1.303 | 71.6 |
| 65.0% | 35.0% | 117.4 | 1734.0 | 83.8 | 1.6 | 1.1 | 6391.7 | 1.314 | 68.6 |
| 70.0% | 30.0% | 112.9 | 1696.7 | 84.6 | 1.6 | 1.1 | 6257.5 | 1.326 | 65.8 |
| 75.0% | 25.0% | 108.5 | 1659.4 | 85.4 | 1.6 | 1.0 | 6123.4 | 1.338 | 63.0 |
| 80.0% | 20.0% | 104.2 | 1622.2 | 86.1 | 1.5 | 0.9 | 5988.5 | 1.350 | 60.4 |
| 85.0% | 15.0% | 100.0 | 1584.8 | 86.9 | 1.4 | 0.8 | 5851.4 | 1.362 | 57.7 |
| 90.0% | 10.0% | 95.7 | 1547.0 | 87.6 | 1.1 | 0.6 | 5707.9 | 1.374 | 55.1 |
| 95.0% | 5.0% | 91.4 | 1508.6 | 88.3 | 0.7 | 0.3 | 5552.7 | 1.386 | 52.5 |
| 100.0% | 0.0% | 86.8 | 1469.0 | 89.1 | 0.0 | 0.0 | 5375.7 | 1.396 | 49.7 |

TABLE 4AB
CF₃NO₂ AND AMMONIA/AIR CONDITIONING TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Ammonia | kPa | kPa | ° C. | Glide, ° C. | Glide, ° C. | kJ/s | COP | kg/s |
| 0.0% | 100.0% | 515.7 | 1782.7 | 139.8 | 0.0 | 0.0 | 43289.2 | 4.118 | 39.2 |
| 5.0% | 95.0% | 511.2 | 1767.7 | 138.5 | 0.2 | 0.2 | 42875.4 | 4.121 | 40.6 |
| 10.0% | 90.0% | 506.6 | 1752.4 | 137.0 | 0.3 | 0.4 | 42453.8 | 4.124 | 42.1 |
| 15.0% | 85.0% | 502.0 | 1736.6 | 135.5 | 0.4 | 0.6 | 42020.6 | 4.128 | 43.7 |
| 20.0% | 80.0% | 497.2 | 1720.2 | 133.8 | 0.5 | 0.8 | 41571.8 | 4.133 | 45.6 |
| 25.0% | 75.0% | 492.3 | 1703.1 | 131.9 | 0.6 | 0.9 | 41103.9 | 4.137 | 47.5 |
| 30.0% | 70.0% | 487.2 | 1685.2 | 129.9 | 0.7 | 1.1 | 40615.0 | 4.143 | 49.8 |
| 35.0% | 65.0% | 481.8 | 1666.4 | 127.7 | 0.8 | 1.2 | 40097.8 | 4.148 | 52.2 |
| 40.0% | 60.0% | 476.2 | 1646.5 | 125.3 | 0.8 | 1.2 | 39549.3 | 4.154 | 55.0 |
| 45.0% | 55.0% | 470.4 | 1625.5 | 122.7 | 0.8 | 1.3 | 38965.0 | 4.161 | 58.1 |
| 50.0% | 50.0% | 464.2 | 1603.1 | 119.8 | 0.8 | 1.3 | 38339.7 | 4.167 | 61.6 |
| 55.0% | 45.0% | 457.7 | 1579.4 | 116.7 | 0.8 | 1.2 | 37668.4 | 4.173 | 65.6 |
| 60.0% | 40.0% | 450.9 | 1554.3 | 113.3 | 0.7 | 1.1 | 36946.1 | 4.179 | 70.3 |
| 65.0% | 35.0% | 443.7 | 1527.8 | 109.5 | 0.6 | 1.0 | 36167.4 | 4.185 | 75.9 |
| 70.0% | 30.0% | 436.4 | 1500.1 | 105.3 | 0.5 | 0.8 | 35332.6 | 4.189 | 82.7 |
| 75.0% | 25.0% | 429.0 | 1471.8 | 100.6 | 0.4 | 0.6 | 34441.7 | 4.191 | 91.0 |
| 80.0% | 20.0% | 422.0 | 1444.1 | 95.5 | 0.2 | 0.4 | 33505.0 | 4.189 | 101.8 |
| 85.0% | 15.0% | 416.2 | 1419.5 | 89.7 | 0.1 | 0.2 | 32551.2 | 4.181 | 116.2 |
| 90.0% | 10.0% | 413.4 | 1403.4 | 83.2 | 0.0 | 0.0 | 31646.5 | 4.160 | 137.1 |
| 95.0% | 5.0% | 417.6 | 1408.5 | 76.0 | 0.1 | 0.0 | 30935.5 | 4.115 | 170.4 |
| 100.0% | 0.0% | 441.0 | 1469.0 | 67.6 | 0.0 | 0.0 | 30771.3 | 4.007 | 234.3 |

TABLE 4AC
CF₃NO₂ AND AMMONIA/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Ammonia | kPa | kPa | ° C. | Glide, ° C. | Glide, ° C. | kJ/s | COP | kg/s |
| 0.0% | 100.0% | 315.1 | 1782.7 | 179.9 | 0.0 | 0.0 | 26785.5 | 2.811 | 24.6 |
| 5.0% | 95.0% | 312.3 | 1767.7 | 177.8 | 0.1 | 0.2 | 26518.7 | 2.814 | 25.5 |
| 10.0% | 90.0% | 309.5 | 1752.4 | 175.5 | 0.3 | 0.4 | 26247.3 | 2.818 | 26.4 |
| 15.0% | 85.0% | 306.6 | 1736.6 | 173.1 | 0.4 | 0.6 | 25968.3 | 2.822 | 27.5 |
| 20.0% | 80.0% | 303.7 | 1720.2 | 170.4 | 0.5 | 0.8 | 25679.5 | 2.826 | 28.6 |
| 25.0% | 75.0% | 300.7 | 1703.1 | 167.6 | 0.6 | 0.9 | 25378.5 | 2.831 | 29.8 |
| 30.0% | 70.0% | 297.6 | 1685.2 | 164.5 | 0.6 | 1.1 | 25062.6 | 2.836 | 31.2 |
| 35.0% | 65.0% | 294.3 | 1666.4 | 161.1 | 0.7 | 1.2 | 24729.4 | 2.841 | 32.8 |
| 40.0% | 60.0% | 290.9 | 1646.5 | 157.5 | 0.7 | 1.2 | 24376.0 | 2.846 | 34.5 |
| 45.0% | 55.0% | 287.4 | 1625.5 | 153.6 | 0.7 | 1.3 | 23999.4 | 2.852 | 36.4 |
| 50.0% | 50.0% | 283.7 | 1603.1 | 149.3 | 0.7 | 1.3 | 23596.3 | 2.857 | 38.6 |
| 55.0% | 45.0% | 279.8 | 1579.4 | 144.6 | 0.7 | 1.2 | 23163.0 | 2.863 | 41.2 |
| 60.0% | 40.0% | 275.7 | 1554.3 | 139.5 | 0.6 | 1.1 | 22696.0 | 2.868 | 44.1 |
| 65.0% | 35.0% | 271.4 | 1527.8 | 133.9 | 0.5 | 1.0 | 22191.8 | 2.872 | 47.7 |
| 70.0% | 30.0% | 267.0 | 1500.1 | 127.7 | 0.4 | 0.8 | 21647.6 | 2.876 | 51.9 |
| 75.0% | 25.0% | 262.7 | 1471.8 | 120.9 | 0.3 | 0.6 | 21062.8 | 2.877 | 57.1 |
| 80.0% | 20.0% | 258.6 | 1444.1 | 113.4 | 0.2 | 0.4 | 20441.0 | 2.874 | 63.9 |
| 85.0% | 15.0% | 255.3 | 1419.5 | 105.0 | 0.0 | 0.2 | 19795.0 | 2.865 | 73.0 |
| 90.0% | 10.0% | 254.0 | 1403.4 | 95.7 | 0.0 | 0.0 | 19155.7 | 2.843 | 86.1 |
| 95.0% | 5.0% | 257.2 | 1408.5 | 85.2 | 0.1 | 0.0 | 18590.4 | 2.798 | 107.1 |
| 100.0% | 0.0% | 272.9 | 1469.0 | 73.3 | 0.0 | 0.0 | 18270.3 | 2.695 | 147.5 |

TABLE AD
CF₃NO₂ AND AMMONIA/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | Ammonia | kPa | kPa | ° C. | Glide, ° C. | Glide, ° C. | kJ/s | COP | kg/s |
| 0.0% | 100.0% | 97.9 | 1782.7 | 283.9 | 0.0 | 0.0 | 8637.6 | 1.505 | 8.2 |
| 5.0% | 95.0% | 97.0 | 1767.7 | 279.8 | 0.1 | 0.2 | 8544.1 | 1.508 | 8.5 |
| 10.0% | 90.0% | 96.1 | 1752.4 | 275.3 | 0.2 | 0.4 | 8449.1 | 1.512 | 8.8 |
| 15.0% | 85.0% | 95.2 | 1736.6 | 270.6 | 0.3 | 0.6 | 8351.7 | 1.516 | 9.2 |
| 20.0% | 80.0% | 94.3 | 1720.2 | 265.5 | 0.3 | 0.8 | 8251.0 | 1.520 | 9.5 |
| 25.0% | 75.0% | 93.4 | 1703.1 | 260.0 | 0.4 | 0.9 | 8146.0 | 1.524 | 10.0 |
| 30.0% | 70.0% | 92.4 | 1685.2 | 254.1 | 0.5 | 1.1 | 8035.8 | 1.528 | 10.4 |
| 35.0% | 65.0% | 91.4 | 1666.4 | 247.7 | 0.5 | 1.2 | 7919.6 | 1.533 | 10.9 |
| 40.0% | 60.0% | 90.3 | 1646.5 | 240.9 | 0.5 | 1.2 | 7796.2 | 1.538 | 11.5 |
| 45.0% | 55.0% | 89.3 | 1625.5 | 233.5 | 0.5 | 1.3 | 7664.5 | 1.542 | 12.2 |
| 50.0% | 50.0% | 88.1 | 1603.1 | 225.5 | 0.5 | 1.3 | 7523.2 | 1.547 | 12.9 |
| 55.0% | 45.0% | 86.9 | 1579.4 | 216.8 | 0.5 | 1.2 | 7370.9 | 1.552 | 13.7 |
| 60.0% | 40.0% | 85.7 | 1554.3 | 207.4 | 0.4 | 1.1 | 7206.1 | 1.556 | 14.7 |
| 65.0% | 35.0% | 84.4 | 1527.8 | 197.1 | 0.4 | 1.0 | 7027.2 | 1.560 | 15.9 |
| 70.0% | 30.0% | 83.1 | 1500.1 | 185.8 | 0.3 | 0.8 | 6832.8 | 1.562 | 17.3 |
| 75.0% | 25.0% | 81.8 | 1471.8 | 173.5 | 0.2 | 0.6 | 6621.3 | 1.563 | 19.1 |
| 80.0% | 20.0% | 80.7 | 1444.1 | 159.9 | 0.1 | 0.4 | 6392.3 | 1.560 | 21.3 |
| 85.0% | 15.0% | 79.8 | 1419.5 | 144.9 | 0.0 | 0.2 | 6146.6 | 1.551 | 24.4 |
| 90.0% | 10.0% | 79.6 | 1403.4 | 128.4 | 0.0 | 0.0 | 5886.9 | 1.530 | 28.8 |
| 95.0% | 5.0% | 80.9 | 1408.5 | 109.9 | 0.1 | 0.0 | 5621.5 | 1.488 | 35.9 |
| 100.0% | 0.0% | 86.8 | 1469.0 | 89.1 | 0.0 | 0.0 | 5375.7 | 1.396 | 49.7 |

TABLE AE
CF₃NO₂ AND HFO-1243zf/AIR CONDITIONING TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1243zf | kPa | kPa | ° C. | Glide, ° C. | Glide, ° C. | kJ/s | COP | kg/s |
| 0.0% | 100.0% | 300.2 | 984.0 | 64.5 | 0.0 | 0.0 | 21426.4 | 4.139 | 130.5 |
| 5.0% | 95.0% | 304.3 | 998.4 | 64.7 | 0.1 | 0.1 | 21717.8 | 4.136 | 133.5 |
| 10.0% | 90.0% | 308.5 | 1013.5 | 64.9 | 0.2 | 0.3 | 22021.8 | 4.132 | 136.5 |
| 15.0% | 85.0% | 313.0 | 1029.3 | 65.0 | 0.3 | 0.4 | 22338.9 | 4.129 | 139.8 |
| 20.0% | 80.0% | 317.7 | 1045.8 | 65.2 | 0.3 | 0.5 | 22669.8 | 4.125 | 143.1 |
| 25.0% | 75.0% | 322.6 | 1063.2 | 65.4 | 0.4 | 0.7 | 23015.2 | 4.121 | 146.7 |
| 30.0% | 70.0% | 327.8 | 1081.4 | 65.6 | 0.5 | 0.8 | 23375.9 | 4.116 | 150.4 |
| 35.0% | 65.0% | 333.3 | 1100.5 | 65.8 | 0.5 | 0.9 | 23752.8 | 4.111 | 154.4 |
| 40.0% | 60.0% | 339.0 | 1120.6 | 66.0 | 0.6 | 1.0 | 24146.8 | 4.106 | 158.5 |
| 45.0% | 55.0% | 345.0 | 1141.7 | 66.1 | 0.6 | 1.0 | 24559.0 | 4.100 | 162.9 |
| 50.0% | 50.0% | 351.4 | 1163.9 | 66.3 | 0.7 | 1.1 | 24991.8 | 4.094 | 167.6 |
| 55.0% | 45.0% | 358.2 | 1187.3 | 66.5 | 0.7 | 1.1 | 25444.3 | 4.087 | 172.5 |
| 60.0% | 40.0% | 365.3 | 1211.9 | 66.7 | 0.7 | 1.2 | 25918.9 | 4.080 | 177.7 |
| 65.0% | 35.0% | 372.9 | 1238.0 | 66.8 | 0.7 | 1.1 | 26417.3 | 4.072 | 183.3 |
| 70.0% | 30.0% | 380.9 | 1265.5 | 67.0 | 0.7 | 1.1 | 26941.3 | 4.064 | 189.2 |
| 75.0% | 25.0% | 389.4 | 1294.6 | 67.1 | 0.6 | 1.0 | 27493.2 | 4.056 | 195.5 |
| 80.0% | 20.0% | 398.4 | 1325.4 | 67.2 | 0.6 | 0.9 | 28074.6 | 4.046 | 202.2 |
| 85.0% | 15.0% | 408.0 | 1358.1 | 67.4 | 0.5 | 0.8 | 28690.8 | 4.037 | 209.4 |
| 90.0% | 10.0% | 418.3 | 1392.8 | 67.5 | 0.3 | 0.6 | 29343.4 | 4.027 | 217.1 |
| 95.0% | 5.0% | 429.3 | 1429.7 | 67.6 | 0.2 | 0.3 | 30035.8 | 4.017 | 225.4 |
| 100.0% | 0.0% | 441.0 | 1469.0 | 67.6 | 0.0 | 0.0 | 30771.3 | 4.007 | 234.3 |

TABLE AF
CF₃NO₂ AND HFO-1243zf/MEDIUM TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1243zf | kPa | kPa | ° C. | Glide, ° C. | Glide, ° C. | kJ/s | COP | kg/s |
| 0.0% | 100.0% | 187.1 | 984.0 | 69.4 | 0.0 | 0.0 | 12854.6 | 2.794 | 83.3 |
| 5.0% | 95.0% | 189.6 | 998.4 | 69.6 | 0.1 | 0.1 | 13022.2 | 2.792 | 85.1 |
| 10.0% | 90.0% | 192.1 | 1013.5 | 69.8 | 0.1 | 0.3 | 13196.5 | 2.789 | 87.0 |
| 15.0% | 85.0% | 194.8 | 1029.3 | 70.0 | 0.2 | 0.4 | 13378.8 | 2.786 | 89.0 |
| 20.0% | 80.0% | 197.6 | 1045.8 | 70.2 | 0.3 | 0.5 | 13569.2 | 2.783 | 91.1 |
| 25.0% | 75.0% | 200.6 | 1063.2 | 70.5 | 0.3 | 0.7 | 13768.2 | 2.780 | 93.3 |
| 30.0% | 70.0% | 203.7 | 1081.4 | 70.7 | 0.4 | 0.8 | 13976.1 | 2.776 | 95.6 |
| 35.0% | 65.0% | 207.0 | 1100.5 | 70.9 | 0.4 | 0.9 | 14193.5 | 2.773 | 98.0 |
| 40.0% | 60.0% | 210.5 | 1120.6 | 71.1 | 0.5 | 1.0 | 14421.4 | 2.769 | 100.6 |
| 45.0% | 55.0% | 214.2 | 1141.7 | 71.3 | 0.5 | 1.0 | 14659.3 | 2.764 | 103.3 |
| 50.0% | 50.0% | 218.1 | 1163.9 | 71.5 | 0.5 | 1.1 | 14908.5 | 2.760 | 106.2 |
| 55.0% | 45.0% | 222.1 | 1187.3 | 71.7 | 0.6 | 1.1 | 15169.9 | 2.754 | 109.2 |
| 60.0% | 40.0% | 226.5 | 1211.9 | 71.9 | 0.6 | 1.2 | 15444.4 | 2.749 | 112.5 |
| 65.0% | 35.0% | 231.1 | 1238.0 | 72.1 | 0.6 | 1.1 | 15732.9 | 2.743 | 115.9 |
| 70.0% | 30.0% | 236.0 | 1265.5 | 72.3 | 0.5 | 1.1 | 16036.8 | 2.737 | 119.5 |
| 75.0% | 25.0% | 241.2 | 1294.6 | 72.5 | 0.5 | 1.0 | 16357.3 | 2.731 | 123.4 |
| 80.0% | 20.0% | 246.7 | 1325.4 | 72.7 | 0.4 | 0.9 | 16696.1 | 2.724 | 127.6 |
| 85.0% | 15.0% | 252.6 | 1358.1 | 72.9 | 0.4 | 0.8 | 17054.8 | 2.717 | 132.1 |
| 90.0% | 10.0% | 258.9 | 1392.8 | 73.0 | 0.3 | 0.6 | 17435.5 | 2.710 | 136.9 |
| 95.0% | 5.0% | 265.7 | 1429.7 | 73.2 | 0.1 | 0.3 | 17840.0 | 2.703 | 142.0 |
| 100.0% | 0.0% | 272.9 | 1469.0 | 73.3 | 0.0 | 0.0 | 18270.3 | 2.695 | 147.5 |

TABLE AG
CF₃NO₂ AND HFO-1243zf/LOW TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1243zf | kPa | kPa | ° C. | Glide, ° C. | Glide, ° C. | kJ/s | COP | kg/s |
| 0.0% | 100.0% | 60.7 | 984.0 | 83.7 | 0.0 | 0.0 | 3889.6 | 1.462 | 28.9 |
| 5.0% | 95.0% | 61.4 | 998.4 | 84.0 | 0.0 | 0.1 | 3934.2 | 1.460 | 29.5 |
| 10.0% | 90.0% | 62.2 | 1013.5 | 84.2 | 0.1 | 0.3 | 3980.8 | 1.458 | 30.1 |
| 15.0% | 85.0% | 63.0 | 1029.3 | 84.5 | 0.1 | 0.4 | 4029.6 | 1.456 | 30.7 |
| 20.0% | 80.0% | 63.8 | 1045.8 | 84.8 | 0.1 | 0.5 | 4080.6 | 1.454 | 31.4 |
| 25.0% | 75.0% | 64.7 | 1063.2 | 85.1 | 0.2 | 0.7 | 4134.0 | 1.452 | 32.1 |
| 30.0% | 70.0% | 65.6 | 1081.4 | 85.4 | 0.2 | 0.8 | 4190.0 | 1.449 | 32.9 |
| 35.0% | 65.0% | 66.6 | 1100.5 | 85.6 | 0.2 | 0.9 | 4248.6 | 1.447 | 33.6 |
| 40.0% | 60.0% | 67.6 | 1120.6 | 85.9 | 0.3 | 1.0 | 4310.0 | 1.444 | 34.5 |
| 45.0% | 55.0% | 68.7 | 1141.7 | 86.2 | 0.3 | 1.0 | 4374.5 | 1.441 | 35.4 |
| 50.0% | 50.0% | 69.8 | 1163.9 | 86.5 | 0.3 | 1.1 | 4442.2 | 1.438 | 36.3 |
| 55.0% | 45.0% | 71.1 | 1187.3 | 86.8 | 0.3 | 1.1 | 4513.4 | 1.435 | 37.3 |
| 60.0% | 40.0% | 72.4 | 1211.9 | 87.1 | 0.3 | 1.2 | 4588.3 | 1.431 | 38.3 |
| 65.0% | 35.0% | 73.8 | 1238.0 | 87.3 | 0.3 | 1.1 | 4667.3 | 1.427 | 39.4 |
| 70.0% | 30.0% | 75.3 | 1265.5 | 87.6 | 0.3 | 1.1 | 4750.8 | 1.423 | 40.6 |
| 75.0% | 25.0% | 76.9 | 1294.6 | 87.9 | 0.3 | 1.0 | 4839.1 | 1.419 | 41.9 |
| 80.0% | 20.0% | 78.6 | 1325.4 | 88.1 | 0.3 | 0.9 | 4932.9 | 1.415 | 43.2 |
| 85.0% | 15.0% | 80.4 | 1358.1 | 88.4 | 0.2 | 0.8 | 5032.8 | 1.410 | 44.7 |
| 90.0% | 10.0% | 82.4 | 1392.8 | 88.6 | 0.2 | 0.6 | 5139.3 | 1.406 | 46.2 |
| 95.0% | 5.0% | 84.5 | 1429.7 | 88.9 | 0.1 | 0.3 | 5253.4 | 1.401 | 47.9 |
| 100.0% | 0.0% | 86.8 | 1469.0 | 89.1 | 0.0 | 0.0 | 5375.7 | 1.396 | 49.7 |

TABLE AH
CF₃NO₂ AND HFO-1261/AIR CONDITIONING TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1261 | kPa | kPa | ° C. | Glide, ° C. | Glide, ° C. | kJ/s | COP | kg/s |
| 0.0% | 100.0% | 295.7 | 921.1 | 72.7 | 0.0 | 0.0 | 21180.9 | 4.248 | 80.0 |
| 5.0% | 95.0% | 311.0 | 966.2 | 73.3 | 1.7 | 2.0 | 22284.1 | 4.266 | 86.2 |
| 10.0% | 90.0% | 325.9 | 1010.9 | 73.7 | 3.0 | 3.6 | 23316.6 | 4.272 | 92.6 |
| 15.0% | 85.0% | 340.4 | 1055.3 | 74.0 | 4.1 | 4.9 | 24282.3 | 4.269 | 99.2 |
| 20.0% | 80.0% | 354.5 | 1099.4 | 74.2 | 4.9 | 5.9 | 25186.4 | 4.256 | 106.1 |
| 25.0% | 75.0% | 368.4 | 1143.5 | 74.3 | 5.3 | 6.5 | 26035.0 | 4.236 | 113.5 |
| 30.0% | 70.0% | 382.3 | 1187.9 | 74.2 | 5.6 | 6.9 | 26836.6 | 4.210 | 121.2 |
| 35.0% | 65.0% | 396.3 | 1232.7 | 74.0 | 5.6 | 7.0 | 27601.1 | 4.179 | 129.5 |
| 40.0% | 60.0% | 410.5 | 1278.2 | 73.6 | 5.4 | 6.9 | 28340.4 | 4.145 | 138.5 |
| 45.0% | 55.0% | 425.3 | 1324.7 | 73.1 | 5.0 | 6.4 | 29068.8 | 4.110 | 148.3 |
| 50.0% | 50.0% | 440.7 | 1372.3 | 72.5 | 4.4 | 5.8 | 29802.3 | 4.074 | 159.2 |
| 55.0% | 45.0% | 457.0 | 1421.0 | 71.7 | 3.7 | 4.9 | 30558.3 | 4.042 | 171.2 |
| 60.0% | 40.0% | 474.1 | 1470.2 | 70.8 | 2.8 | 3.8 | 31351.1 | 4.015 | 184.5 |
| 65.0% | 35.0% | 491.9 | 1518.7 | 69.8 | 1.8 | 2.7 | 32182.3 | 3.998 | 199.2 |
| 70.0% | 30.0% | 508.9 | 1563.5 | 68.7 | 0.9 | 1.5 | 33005.5 | 3.990 | 214.7 |
| 75.0% | 25.0% | 521.8 | 1600.0 | 67.8 | 0.2 | 0.6 | 33668.3 | 3.985 | 229.3 |
| 80.0% | 20.0% | 525.6 | 1622.3 | 67.5 | 0.0 | 0.1 | 33927.1 | 3.968 | 240.2 |
| 85.0% | 15.0% | 518.6 | 1624.8 | 67.7 | 0.3 | 0.0 | 33737.9 | 3.948 | 245.9 |
| 90.0% | 10.0% | 502.7 | 1603.7 | 68.1 | 0.8 | 0.3 | 33237.4 | 3.949 | 247.1 |
| 95.0% | 5.0% | 478.2 | 1554.9 | 68.1 | 1.0 | 0.6 | 32404.0 | 3.978 | 244.1 |
| 100.0% | 0.0% | 441.0 | 1469.0 | 67.6 | 0.0 | 0.0 | 30771.3 | 4.007 | 234.3 |

TABLE 4AI
CF₃NO₂ AND HFO-1261/MEDIUM TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1261 | kPa | kPa | ° C. | Glide, ° C. | Glide, ° C. | kJ/s | COP | kg/s |
| 0.0% | 100.0% | 188.9 | 921.1 | 80.9 | 0.0 | 0.0 | 13252.8 | 2.909 | 52.5 |
| 5.0% | 95.0% | 198.9 | 966.2 | 81.2 | 1.6 | 2.0 | 13941.6 | 2.920 | 56.5 |
| 10.0% | 90.0% | 208.5 | 1010.9 | 81.5 | 2.8 | 3.6 | 14578.7 | 2.922 | 60.7 |
| 15.0% | 85.0% | 217.7 | 1055.3 | 81.6 | 3.8 | 4.9 | 15167.6 | 2.917 | 65.0 |
| 20.0% | 80.0% | 226.7 | 1099.4 | 81.7 | 4.4 | 5.9 | 15712.5 | 2.906 | 69.5 |
| 25.0% | 75.0% | 235.6 | 1143.5 | 81.6 | 4.9 | 6.5 | 16218.2 | 2.890 | 74.2 |
| 30.0% | 70.0% | 244.4 | 1187.9 | 81.3 | 5.1 | 6.9 | 16691.1 | 2.870 | 79.2 |
| 35.0% | 65.0% | 253.2 | 1232.7 | 81.0 | 5.0 | 7.0 | 17138.3 | 2.846 | 84.6 |
| 40.0% | 60.0% | 262.3 | 1278.2 | 80.5 | 4.8 | 6.9 | 17568.6 | 2.820 | 90.4 |
| 45.0% | 55.0% | 271.7 | 1324.7 | 79.9 | 4.4 | 6.4 | 17992.3 | 2.793 | 96.8 |
| 50.0% | 50.0% | 281.6 | 1372.3 | 79.1 | 3.9 | 5.8 | 18421.4 | 2.766 | 103.8 |
| 55.0% | 45.0% | 292.3 | 1421.0 | 78.1 | 3.2 | 4.9 | 18869.1 | 2.741 | 111.6 |
| 60.0% | 40.0% | 303.6 | 1470.2 | 77.0 | 2.4 | 3.8 | 19347.3 | 2.719 | 120.3 |
| 65.0% | 35.0% | 315.6 | 1518.7 | 75.7 | 1.5 | 2.7 | 19859.9 | 2.704 | 130.0 |
| 70.0% | 30.0% | 327.2 | 1563.5 | 74.4 | 0.7 | 1.5 | 20373.7 | 2.696 | 140.2 |
| 75.0% | 25.0% | 335.6 | 1600.0 | 73.4 | 0.1 | 0.6 | 20753.4 | 2.689 | 149.7 |
| 80.0% | 20.0% | 336.2 | 1622.3 | 73.1 | 0.1 | 0.1 | 20774.0 | 2.670 | 155.8 |
| 85.0% | 15.0% | 328.9 | 1624.8 | 73.5 | 0.5 | 0.0 | 20473.0 | 2.650 | 158.0 |
| 90.0% | 10.0% | 316.3 | 1603.7 | 73.8 | 1.0 | 0.3 | 20018.3 | 2.650 | 157.7 |
| 95.0% | 5.0% | 298.8 | 1554.9 | 73.9 | 1.1 | 0.6 | 19397.3 | 2.672 | 154.8 |
| 100.0% | 0.0% | 272.9 | 1469.0 | 73.3 | 0.0 | 0.0 | 18270.3 | 2.695 | 147.5 |

TABLE 4AJ
CF₃NO₂ AND HFO-1261/LOW TEMPERATURE CONDITIONS

| | wt % | | | | | | | | Mass |
|---|---|---|---|---|---|---|---|---|---|
| Wt % | HFO- | P_evap, | P_cond, | T_comp_exit, | Evap | Cond | Capacity, | | Flow, |
| CF3NO2 | 1261 | kPa | kPa | ° C. | Glide, ° C. | Glide, ° C. | kJ/s | COP | kg/s |
| 0.0% | 100.0% | 65.7 | 921.1 | 102.8 | 0.0 | 0.0 | 4465.6 | 1.575 | 19.6 |
| 5.0% | 95.0% | 69.3 | 966.2 | 102.7 | 1.3 | 2.0 | 4697.1 | 1.579 | 21.1 |
| 10.0% | 90.0% | 72.7 | 1010.9 | 102.5 | 2.3 | 3.6 | 4905.7 | 1.578 | 22.7 |
| 15.0% | 85.0% | 75.9 | 1055.3 | 102.2 | 3.1 | 4.9 | 5093.4 | 1.573 | 24.3 |
| 20.0% | 80.0% | 79.0 | 1099.4 | 101.9 | 3.6 | 5.9 | 5262.3 | 1.564 | 25.9 |
| 25.0% | 75.0% | 82.0 | 1143.5 | 101.4 | 3.9 | 6.5 | 5414.9 | 1.553 | 27.6 |
| 30.0% | 70.0% | 85.0 | 1187.9 | 100.8 | 4.0 | 6.9 | 5554.1 | 1.539 | 29.5 |
| 35.0% | 65.0% | 88.1 | 1232.7 | 100.1 | 3.9 | 7.0 | 5682.9 | 1.523 | 31.4 |
| 40.0% | 60.0% | 91.2 | 1278.2 | 99.2 | 3.7 | 6.9 | 5805.3 | 1.505 | 33.5 |
| 45.0% | 55.0% | 94.5 | 1324.7 | 98.1 | 3.3 | 6.4 | 5925.8 | 1.487 | 35.8 |
| 50.0% | 50.0% | 98.0 | 1372.3 | 96.9 | 2.9 | 5.8 | 6049.4 | 1.468 | 38.4 |
| 55.0% | 45.0% | 101.9 | 1421.0 | 95.4 | 2.3 | 4.9 | 6182.4 | 1.450 | 41.3 |
| 60.0% | 40.0% | 106.1 | 1470.2 | 93.8 | 1.6 | 3.8 | 6331.1 | 1.435 | 44.6 |
| 65.0% | 35.0% | 110.8 | 1518.7 | 91.9 | 1.0 | 2.7 | 6497.8 | 1.422 | 48.3 |
| 70.0% | 30.0% | 115.4 | 1563.5 | 90.0 | 0.3 | 1.5 | 6668.9 | 1.414 | 52.3 |
| 75.0% | 25.0% | 117.8 | 1600.0 | 88.7 | 0.0 | 0.6 | 6736.0 | 1.402 | 55.5 |
| 80.0% | 20.0% | 115.1 | 1622.3 | 89.1 | 0.3 | 0.1 | 6560.2 | 1.378 | 56.2 |
| 85.0% | 15.0% | 110.1 | 1624.8 | 89.8 | 0.8 | 0.0 | 6318.8 | 1.363 | 55.8 |
| 90.0% | 10.0% | 104.2 | 1603.7 | 90.1 | 1.3 | 0.3 | 6082.1 | 1.364 | 54.9 |
| 95.0% | 5.0% | 97.1 | 1554.9 | 89.8 | 1.4 | 0.6 | 5822.3 | 1.381 | 53.2 |
| 100.0% | 0.0% | 86.8 | 1469.0 | 89.1 | 0.0 | 0.0 | 5375.7 | 1.396 | 49.7 |

TABLE 4AK
CF₃NO₂ AND CO2/AIR CONDITIONING TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | CO2 | kPa | kPa | ° C. | Glide, ° C. | Glide, ° C. | kJ/s | COP | kg/s |
| 100.0% | 0.0% | 441.0 | 1469.0 | 67.6 | 0.0 | 0.0 | 30771.3 | 4.007 | 234.3 |
| 99.5% | 0.5% | 466.1 | 1526.9 | 68.7 | 2.4 | 1.9 | 32684.1 | 4.087 | 245.4 |
| 99.0% | 1.0% | 490.2 | 1583.7 | 69.7 | 4.6 | 3.6 | 34497.1 | 4.152 | 255.7 |
| 98.5% | 1.5% | 513.3 | 1639.3 | 70.7 | 6.5 | 5.1 | 36211.4 | 4.206 | 265.4 |
| 98.0% | 2.0% | 535.4 | 1693.7 | 71.6 | 8.2 | 6.5 | 37830.7 | 4.248 | 274.4 |
| 97.5% | 2.5% | 556.5 | 1747.1 | 72.5 | 9.7 | 7.8 | 39360.1 | 4.282 | 282.9 |
| 97.0% | 3.0% | 576.9 | 1799.5 | 73.3 | 11.0 | 9.0 | 40803.3 | 4.307 | 290.8 |
| 96.5% | 3.5% | 596.4 | 1850.9 | 74.1 | 12.2 | 10.1 | 42168.8 | 4.324 | 298.2 |
| 96.0% | 4.0% | 615.3 | 1901.3 | 74.9 | 13.3 | 11.1 | 43461.9 | 4.336 | 305.2 |
| 95.5% | 4.5% | 633.6 | 1950.9 | 75.7 | 14.3 | 12.0 | 44687.5 | 4.343 | 311.8 |
| 95.0% | 5.0% | 651.3 | 1999.7 | 76.4 | 15.2 | 12.9 | 45851.2 | 4.345 | 318.0 |
| 94.5% | 5.5% | 668.5 | 2047.7 | 77.1 | 16.0 | 13.6 | 46957.8 | 4.344 | 323.9 |
| 94.0% | 6.0% | 685.2 | 2095.0 | 77.8 | 16.7 | 14.3 | 48011.9 | 4.339 | 329.5 |
| 93.5% | 6.5% | 701.5 | 2141.7 | 78.4 | 17.3 | 15.0 | 49017.7 | 4.331 | 334.9 |
| 93.0% | 7.0% | 717.5 | 2187.8 | 79.1 | 17.9 | 15.5 | 49979.1 | 4.321 | 340.0 |
| 92.5% | 7.5% | 733.1 | 2233.3 | 79.7 | 18.4 | 16.1 | 50899.7 | 4.309 | 344.8 |
| 92.0% | 8.0% | 748.4 | 2278.4 | 80.3 | 18.9 | 16.6 | 51782.7 | 4.295 | 349.5 |
| 91.5% | 8.5% | 763.4 | 2323.0 | 80.9 | 19.4 | 17.0 | 52631.2 | 4.280 | 354.0 |
| 91.0% | 9.0% | 778.1 | 2367.1 | 81.4 | 19.7 | 17.4 | 53447.8 | 4.263 | 358.3 |
| 90.5% | 9.5% | 792.6 | 2410.9 | 82.0 | 20.1 | 17.8 | 54234.9 | 4.246 | 362.5 |
| 90.0% | 10.0% | 806.9 | 2454.3 | 82.5 | 20.4 | 18.1 | 54995.0 | 4.227 | 366.5 |

TABLE AL
CF₃NO₂ AND CO2/MEDIUM TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | CO2 | kPa | kPa | ° C. | Glide, ° C. | Glide, ° C. | kJ/s | COP | kg/s |
| 100.0% | 0.0% | 272.9 | 1469.0 | 73.3 | 0.0 | 0.0 | 18270.3 | 2.695 | 147.5 |
| 99.5% | 0.5% | 291.0 | 1526.9 | 74.3 | 2.6 | 1.9 | 19586.5 | 2.755 | 155.7 |
| 99.0% | 1.0% | 308.1 | 1583.7 | 75.3 | 4.9 | 3.6 | 20823.9 | 2.804 | 163.2 |
| 98.5% | 1.5% | 324.3 | 1639.3 | 76.3 | 6.9 | 5.1 | 21984.6 | 2.843 | 170.1 |
| 98.0% | 2.0% | 339.7 | 1693.7 | 77.3 | 8.7 | 6.5 | 23071.6 | 2.874 | 176.5 |
| 97.5% | 2.5% | 354.3 | 1747.1 | 78.2 | 10.2 | 7.8 | 24091.0 | 2.898 | 182.5 |
| 97.0% | 3.0% | 368.2 | 1799.5 | 79.1 | 11.6 | 9.0 | 25047.4 | 2.916 | 188.0 |
| 96.5% | 3.5% | 381.5 | 1850.9 | 80.0 | 12.8 | 10.1 | 25945.7 | 2.929 | 193.1 |
| 96.0% | 4.0% | 394.2 | 1901.3 | 80.9 | 13.9 | 11.1 | 26791.4 | 2.938 | 197.9 |
| 95.5% | 4.5% | 406.5 | 1950.9 | 81.7 | 14.8 | 12.0 | 27588.4 | 2.943 | 202.3 |
| 95.0% | 5.0% | 418.3 | 1999.7 | 82.6 | 15.7 | 12.9 | 28341.2 | 2.944 | 206.5 |
| 94.5% | 5.5% | 429.7 | 2047.7 | 83.4 | 16.4 | 13.6 | 29053.6 | 2.943 | 210.5 |
| 94.0% | 6.0% | 440.7 | 2095.0 | 84.2 | 17.1 | 14.3 | 29729.0 | 2.940 | 214.2 |
| 93.5% | 6.5% | 451.4 | 2141.7 | 84.9 | 17.8 | 15.0 | 30370.8 | 2.935 | 217.7 |
| 93.0% | 7.0% | 461.9 | 2187.8 | 85.7 | 18.3 | 15.5 | 30981.9 | 2.928 | 221.1 |
| 92.5% | 7.5% | 472.0 | 2233.3 | 86.4 | 18.8 | 16.1 | 31564.8 | 2.919 | 224.2 |
| 92.0% | 8.0% | 482.0 | 2278.4 | 87.1 | 19.3 | 16.6 | 32122.1 | 2.909 | 227.3 |
| 91.5% | 8.5% | 491.7 | 2323.0 | 87.8 | 19.7 | 17.0 | 32655.9 | 2.899 | 230.2 |
| 91.0% | 9.0% | 501.2 | 2367.1 | 88.5 | 20.0 | 17.4 | 33168.1 | 2.887 | 232.9 |
| 90.5% | 9.5% | 510.6 | 2410.9 | 89.2 | 20.4 | 17.8 | 33662.0 | 2.875 | 235.6 |
| 90.0% | 10.0% | 519.8 | 2454.3 | 89.8 | 20.7 | 18.1 | 34136.5 | 2.862 | 238.2 |

TABLE 4AM
CF₃NO₂ AND CO2/LOW TEMPERATURE CONDITIONS

| | | | | | | | | | Mass |
|---|---|---|---|---|---|---|---|---|---|
| Wt % | wt % | P_evap, | P_cond, | T_comp_exit, | Evap | Cond | Capacity, | | Flow, |
| CF3NO2 | CO2 | kPa | kPa | ° C. | Glide, ° C. | Glide, ° C. | kJ/s | COP | kg/s |
| 100.0% | 0.0% | 86.8 | 1469.0 | 89.1 | 0.0 | 0.0 | 5375.7 | 1.396 | 49.7 |
| 99.5% | 0.5% | 95.1 | 1526.9 | 89.8 | 3.2 | 1.9 | 5936.6 | 1.437 | 53.8 |
| 99.0% | 1.0% | 102.8 | 1583.7 | 90.7 | 5.8 | 3.6 | 6451.8 | 1.470 | 57.5 |
| 98.5% | 1.5% | 109.8 | 1639.3 | 91.6 | 8.0 | 5.1 | 6924.4 | 1.496 | 60.8 |
| 98.0% | 2.0% | 116.4 | 1693.7 | 92.6 | 9.9 | 6.5 | 7358.2 | 1.516 | 63.7 |
| 97.5% | 2.5% | 122.4 | 1747.1 | 93.7 | 11.5 | 7.8 | 7757.5 | 1.531 | 66.4 |
| 97.0% | 3.0% | 128.1 | 1799.5 | 94.7 | 12.9 | 9.0 | 8125.5 | 1.542 | 68.8 |
| 96.5% | 3.5% | 133.4 | 1850.9 | 95.7 | 14.1 | 10.1 | 8465.7 | 1.550 | 71.0 |
| 96.0% | 4.0% | 138.4 | 1901.3 | 96.8 | 15.1 | 11.1 | 8781.1 | 1.556 | 73.0 |
| 95.5% | 4.5% | 143.2 | 1950.9 | 97.8 | 16.0 | 12.0 | 9074.4 | 1.559 | 74.8 |
| 95.0% | 5.0% | 147.7 | 1999.7 | 98.9 | 16.8 | 12.9 | 9347.9 | 1.560 | 76.5 |
| 94.5% | 5.5% | 151.9 | 2047.7 | 99.9 | 17.5 | 13.6 | 9603.6 | 1.559 | 78.1 |
| 94.0% | 6.0% | 156.1 | 2095.0 | 100.9 | 18.1 | 14.3 | 9843.4 | 1.557 | 79.5 |
| 93.5% | 6.5% | 160.0 | 2141.7 | 102.0 | 18.7 | 15.0 | 10069.1 | 1.554 | 80.9 |
| 93.0% | 7.0% | 163.8 | 2187.8 | 103.0 | 19.2 | 15.5 | 10281.9 | 1.550 | 82.2 |
| 92.5% | 7.5% | 167.5 | 2233.3 | 104.0 | 19.6 | 16.1 | 10483.2 | 1.545 | 83.4 |
| 92.0% | 8.0% | 171.1 | 2278.4 | 105.0 | 20.0 | 16.6 | 10674.1 | 1.540 | 84.5 |
| 91.5% | 8.5% | 174.5 | 2323.0 | 106.0 | 20.3 | 17.0 | 10855.6 | 1.534 | 85.5 |
| 91.0% | 9.0% | 177.9 | 2367.1 | 106.9 | 20.6 | 17.4 | 11028.6 | 1.528 | 86.6 |
| 90.5% | 9.5% | 181.2 | 2410.9 | 107.9 | 20.9 | 17.8 | 11193.4 | 1.521 | 87.5 |
| 90.0% | 10.0% | 184.4 | 2454.3 | 108.9 | 21.1 | 18.1 | 11351.7 | 1.513 | 88.4 |

### Example 5-Polyol Foams

This example illustrates the use of blowing agent in accordance with certain preferred embodiments of the present invention, namely the use of each of the compositions identified in Tables 4A-4AL as a blowing agent in the production of polyol foams in accordance with the present invention. The components of a polyol foam formulation are prepared in accordance with the following Table 5:

TABLE 5

| | PBW |
|---|---|
| Polyol component | |
| Voranol 490 | 50 |
| Voranol 391 | 50 |
| Water | 0.5 |
| B-8462 (surfactant) | 2.0 |
| Polycat 8 | 0.3 |
| Polycat 41 | 3.0 |
| BLOWING AGENT | 35 |
| Total | 140.8 |
| Isocyanate | |
| M-20S | 123.8 Index 1.10 |

*Voranol 490 is a sucrose-based polyol and Voranol 391 is a toluene diamine based polyol, and each are from Dow Chemical. B-8462 is a surfactant available from Degussa-Goldschmidt. Polycat catalysts are tertiary amine based and are available from Air Products. Isocyanate M-20S is a product of Bayer LLC.

Each foam is prepared by first mixing the ingredients thereof, but without the addition of blowing agent. Two Fisher-Porter tubes are each filled with about 52.6 grams of the polyol mixture (without blowing agent) and sealed and placed in a refrigerator to cool and form a slight vacuum. Using gas burets, about 17.4 grams of each composition is added to each tube, and the tubes are then placed in an ultrasound bath in warm water and allowed to sit for 30 minutes. The isocyanate mixture, about 87.9 grams, is placed into a metal container and placed in a refrigerator and allowed to cool to about 50° F. The polyol tubes were then opened and weighed into a metal mixing container (about 100 grams of polyol blend are used). The isocyanate from the cooled metal container is then immediately poured into the polyol and mixed with an air mixer with double propellers at 3000 RPM's for 10 seconds. The blend immediately begins to froth with the agitation and is then poured into an 8×8×4 inch box and allowed to foam. The foam is then cut to samples suitable for measuring physical properties and is found to have acceptable density values and K-factors.

### Example 6-Polstyrene Foam

This example illustrates the use of blowing agent in accordance with certain preferred embodiments of the present invention, namely the use of each of the compositions identified in Tables 4A-4AL as a blowing agent in the production of polystyrene foam. A testing apparatus and protocol has been established as an aid to determining whether a specific blowing agent and polymer are capable of producing a foam and the quality of the foam. Ground polymer (Dow Polystyrene 685D) and blowing agent consisting essentially of each composition of the invention is combined in a vessel. The vessel volume is 200 cm³ and it is made from two pipe flanges and a section of 2-inch diameter schedule 40 stainless steel pipe 4 inches long. The vessel is placed in an oven, with temperature set at from about 190° F. to about 285° F., preferably for polystyrene at 265° F., and remains there until temperature equilibrium is reached. The pressure in the vessel is then released, quickly producing a foamed polymer. The blowing agent plasticizes the polymer as it dissolves into it. The resulting density of the two foams thus produced using this method are acceptable.

### Example 7-Extruded Foam

This example demonstrates the performance of each of the compositions identified in Tables 4A-4AL as a blowing agent in polystyrene foam formed in a twin screw type extruder. The apparatus employed in this example is a Leistritz twin screw extruder having the following characteristics: 30 mm co-rotating screws; and L:D Ratio=40:1. The extruder is divided into 10 sections, each representing a L:D of 4:1. The polystyrene resin was introduced into the first section, the blowing agent was introduced into the sixth section, with the extrudate exiting the tenth section. The extruder operated primarily as a melt/mixing extruder. A subsequent cooling extruder is connected in tandem, for which the design characteristics were: Leistritz twin screw extruder; 40 mm co-rotating screws; L:D Ratio=40:1; and Die: 5.0 mm circular. Polystyrene resin, namely Nova Chemical-general extrusion grade polystyrene, identified as Nova 1600, is feed to the extruder under the conditions indicated above. The resin has a recommended melt temperature of 375° F.-525° F. The pressure of the extruder at the die is about 1320 pounds per square inch (psi), and the temperature at the die is about 115° C. A series of blowing agents corresponding to each of the compositions in the Tables above is added to the extruder at the location indicated above, with about 0.5% by weight of talc being included, on the basis of the total blowing agent, as a nucleating agent. Foam is produced using the blowing agent at concentrations of 10% by weight, 12% by weight, and 14% by weight, in accordance with the present invention. The density of the foam produced is in an acceptable range, with a cell size of that is acceptable. Each foam is visually of very good quality, very fine cell size, with no visible or apparent blow holes or voids.

It is apparent that many modifications and variations of this invention as hereinabove set forth may be made without departing from the spirit and scope thereof. The specific embodiments are given by way of example only and the invention is limited only by the terms of the appended claims.

We claim:

1. A composition for use as a blowing agent, foam, foamable composition, foam pre-mixe, solvent, cleaning fluid, extractant, flame retardant, fire suppression agent, deposition agent, propellant, sprayable composition or deposition agent comprising trifluoronitromethane (CF₃NO₂).

2. A composition for use as a heat transfer fluid, blowing agent, foam, foamable composition, foam pre-mixe, solvent, cleaning fluid, extractant, flame retardant, fire suppression agent, deposition agent, propellant, sprayable composition or deposition agent comprising trifluoronitromethane (CF₃NO₂) and at least one adjuvant.

3. The composition of claim 2 wherein said adjuvant comprises at least one co-agent.

4. The composition of claim 2 comprising further comprising from about 1 to 50% by weight of at least one lubricant selected from polyol esters (POEs), capped or uncapped polyalkylene glycols (PAGs), mineral oils, silicone oils, polyvinyl ethers (PVE) oils, and combinations of any two or more of these.

5. The composition of any one or more of claims 2 and 4 wherein said adjuvant comprises a co-refrigerant selected from the group consisting of carbon dioxide (CO₂); tetra- through penta- halogenated C3-C5 olefins; C1-C4 hydrocarbons, hydrofluorocarbons (HFCs); ammonia; and combinations of any two or more of these.

6. A heat transfer fluid comprising from about 1 to about 40 percent by weight of carbon dioxide (CO₂) and from about 99 to about 60 percent by weight of trifluoronitromethane (CF₃NO₂), said fluid having a vapor pressure of at least about 30 psia at 35° F.

7. A method for changing the heat content of a body comprising proving a fluid in accordance with any one of claims 1-6 and transferring heat between said fluid and said body.

8. An improved heat transfer system comprising one or more vessels for evaporating and condensing and a heat transfer fluid contained in one or more of said vessels comprising from about 1 to about 99 percent by weight of trifluoronitromethane (CF₃NO₂) and from about 1 to about 99 percent by weight of at least one co-agent.

9. A non-flammable fluid consisting essentially of trans-1,1,1,3-tetrafluoropropene (HFO-1234ze) and carbon dioxide (CO₂).

10. A sprayable composition comprising a material to be sprayed and a propellant comprising a composition of claim 1.
